(19) United States
(12) Patent Application Publication
(10) Pub. No.: US 2019/0225647 A1
(43) Pub. Date: Jul. 25, 2019

(54) ANTIGENIC PEPTIDES FOR THE DIAGNOSIS, THERAPY MONITORING AND/OR TREATMENT OF PSORIASIS VULGARIS

(71) Applicant: Ludwig-Maximilians-Universität München, Muenchen (DE)

(72) Inventors: Jörg Christoph PRINZ, Starnberg (DE); Klaus DORNMAIR, Olching (DE)

(21) Appl. No.: 15/580,382

(22) PCT Filed: Jun. 30, 2016
(86) PCT No.: PCT/EP2016/065416

§ 371 Date: Dec. 7, 2017

(30) Foreign Application Priority Data
Jul. 3, 2015 (EP) 15175204.5

(51) Int. Cl. C07K 7/06; G01N 33/50; G01N 33/564; G01N 33/569; C07K 14/74; C07K 16/28; C12N 15/113

(52) U.S. Cl. CPC C07K 7/06; G01N 33/505; G01N 33/564; A61K 38/00; C07K 14/70539; C07K 16/2833; C12N 15/113; G01N 33/56977

(57) ABSTRACT

The present invention relates to peptides with a conserved amino acid motif and pharmaceutical compositions comprising these peptides. The present invention further relates to the use of the peptides as biomarker, the medical use of the peptides, in particular in the diagnosis, prevention, monitoring and/or treatment of Psoriasis. The present invention relates to complexes of the peptides of the invention with HLA-C monomers or multimers and their use as biomarker and their medical uses, in particular in the treatment of Psoriasis and in monitoring such treatment. The present invention furthermore relates to means and methods for the prevention and/or treatment of Psoriasis, that comprise inhibiting or blocking the interaction of TCR and HLA-C.

The present invention relates to peptides with a conserved amino acid motif and pharmaceutical compositions comprising these peptides. The present invention further relates to the use of the peptides as biomarker, the medical use of the peptides, in particular in the diagnosis, prevention, monitoring and/or treatment of Psoriasis. The present invention relates to complexes of the peptides of the invention with HLA-C monomers or multimers and their use as biomarker and their medical uses, in particular in the treatment of Psoriasis and in monitoring such treatment. The present invention furthemiore relates to means and methods for the prevention and/or treatment of Psoriasis, that comprise inhibiting or blocking the interaction of TCR and HLA-C.

### BACKGROUND OF THE INVENTION

Biochemical interactions between peptide epitope, specific membrane molecules encoded by the Major Histocompatibility Complex (MHC, in humans human leukocyte antigens HLA) and T-cell antigen receptors (TCR) are required to elicit specific immune responses. This requires activation of T cells by presentation to the T cells of peptides against which a T-cell response should be raised. The peptides are presented to the T-cells by HLA-molecules. T-cells express a clonotypic TCR on the surface. This receptor enables the T-cell to recognize peptide antigens bound to HLA molecules in man. Depending on the type of antigen, being intracellular or extracellular, the antigenic peptides are bound to MHC class I or MHC class II, respectively. The two classes of HLA complexes are recognized by different subsets of T-cells; cytotoxic CD8⁺ T-cells recognizing HLA-class I and CD4⁺ helper cells recognizing HLA-class II. In general, TCR recognition of MHC-peptide complexes results in T-cell activation, clonal expansion and differentiation of the T-cells into effector, memory and regulatory T-cells.

Several autoimmune diseases are associated with particular HLA-class I alleles. Deciphering the specificity of autoimmune responses and understanding the role of the human leukocyte antigen (HLA) region in autoimmune diseases, with different allelic variants associated with each disease, are important unmet medical needs. Neither the functional implications of the HLA associations nor specific antigens of autoreactive T-cell responses have been determined with certainty. This is due to the complexity of antigen-presentation and TCR-antigen recognition. Antigens of CD8⁺ T cells are short peptides of 8-10 amino acids that are cleaved from intracellular proteins by the proteasome and loaded onto HLA-class I molecules. The peptide-HLA-class complex is then transported onto the cell surface for cognate antigen recognition by αβ TCRs of CD8⁺ T cells (Neefjes et al., 2011). αβ TCRs are heterodimeric molecules composed of an α- and β-chain that are generated by somatic recombination of diverse gene segments and allow for a huge TCR diversity (Davis and Bjorkman, 1988). Approaches to characterize targets of autoimmune responses have to identify and utilize the relevant pathogenic autoreactive TCRs; determine, which HLA molecule of each two HLA-A, -B or -C alleles actually restricts the autoreactive T-cell response; define the autoimmune target cell type within the affected tissue; and provide a test system for the reliable identification and verification of the appropriate autoantigenic TCR peptide ligand. In their entirety, the challenges associated herewith are hard to master.

Psoriasis, or more precisely psoriasis vulgaris is a frequent immune-mediated HLA-associated inflammatory skin disease affecting as presumed, 120 to 180 million people worldwide (Teen et al., 2009; Parisi et al., 2013). Psoriasis has various clinical manifestations, including but not limited to chronic plaque psoriasis, acute guttate psoriasis, localized or generalized pustular psoriasis, psoriatic arthritis, psoriatic nail disease and others. Psoriatic skin lesions are characterized by an excessive inflammatory hyperplasia of the epidermis, which leads to the formation of typical red and scaly plaques (Griffiths and Barker, 2007). Many psoriasis features are consistent with a T-cell mediated autoimmune pathogenesis (Davidson and Diamond, 2001). Like other autoimmune diseases (Uchansk-Ziegler et al., 2012), psoriasis is linked to a particular HLA class I allele: The HLA-class I allele, HLA-C*06:02, on psoriasis susceptibility 1 locus (PSORS1, [MIM177900]) is a major psoriasis risk gene (Nair et al., 2006). The formation of novel psoriatic lesions depends on epidermal influx (Conrad et al., 2007) and clonal expansion of CD8⁺ T cells (Kim et al., 2012; Chang et al., 1997), which cause the epidermal changes by a T helper/suppressor (Th/Ts) 17-like effector phenotype (DiCesare et al., 2009). The most efficient treatments for psoriasis are immunosuppressive, interfere with T-cell recruitment and activation, or neutralize Th17-related cytokines (Griffiths and Barker, 2007; Chiricozzi and Krueger, 2013). Nevertheless, the mechanisms that lead to an organ-specific pathogenic T-cell response in psoriasis are unknown and the autoimmune nature of psoriasis remains hypothetical to date.

US 2008/0025951 A1 discloses the protein INSP052BC, an immunoglobulin domain-containing cell surface recognition molecule, and the use of this protein and nucleic acid sequence from the encoding gene in the diagnosis, prevention and treatment of disease in particular those related to the excessive expression and/or secretion of cytokines. The polypeptide INSP052 comprises the hepacam sequence SRASRALRL.

WO 00/43511 A1 discloses CASB612 polypeptides and polynucleotides and methods for utilizing CASB612 polypeptides and polynucleotides in diagnostics, and vaccines for prophylactic and therapeutic treatment of cancers, particularly ovarian and colon cancers, autoimmune diseases, and related conditions.

WO 2013/104804 A2 discloses a set of polypeptides whereby this set comprises two polypeptides each of which comprises a targeting moiety “T” binding to an antigen “A” and a fragment of “F” of a functional domain, particularly an isolated V_{L} domain (as “F1”) and an isolated V_{F} domain (as “F2”) of an antibody, wherein said two polypeptides are not associated with each other in absence of a substrate/target cell that has “A”, such as HLA-Cw6, on its surface and wherein only upon dimerization of “F1” and “F2” the resulting dimer becomes functional. Furthermore, medical and diagnostic uses of said set are described, whereby the functional dimer results in eliminating and killing the target cells.

Thus, there is a need in the art for improved means and methods for diagnosing and treating Psoriasis, in particular for monitoring the treatment.

### SUMMARY OF THE INVENTION

According to the present invention this object is solved by a a peptide comprising an amino acid sequence of the general formula I

X1ₙ-Arg-X2-X3-Y1-X4-Y2-Arg-Z (I)
wherein
X1 is selected from Phe, Arg, Gly, Met, Ala, Ser, Leu or Val;
n is 0 or 1.
X2 is selected from any amino acid;
X3 is selected from any amino acid;
X4 is selected from Thr, Tyr, Val, Cys, Ser, Ala;
Y1 is selected from Arg, Lys, Gln, or Asn;
Y2 is selected from Arg, Leu or Ser; and
Z is selected from Leu, Met, Ile or Val;
   or
wherein
X1 is selected from Phe, Arg, Gly, Met, Ala, Ser, Leu, Val, His, Tyr, Lys or Trp;
n is 0 or 1.
X2 is selected from any amino acid;
X3 is selected from any amino acid;
X4 is selected from Thr, Tyr, Val, Cys, Ser, Ala, Gly or Arg;
Y1 is selected from Arg, Lys, Gln, or Asn;
Y2 is selected from Arg, Leu or Ser; and
Z is selected from Leu, Met, Ile, Val, Thr or Tyr.

According to the present invention this object is solved by a peptide-HLA-C monomer complex,

comprising

(a) at least one peptide selected from a peptide of the present invention or a peptide comprising or consisting of an amino acid sequence of SEQ ID Nos. 14-16,

(b) a HLA-C monomer binding to T-cell receptor(s) (TCR(s)).

According to the present invention this object is solved by a peptide-HLA-C multimer complex,

comprising a HLA-C multimer which comprises at least two HLA-C monomers (b) and which each comprises at least one peptide of the present invention.

According to the present invention this object is solved by a pharmaceutical composition, comprising

(i) at least one peptide of the present invention and/or a HLA-C monomer as defined herein;
or a peptide-HLA-C monomer complex of the present invention,
or a peptide-HLA-C multimer complex of the present invention,

(ii) optionally, a pharmaceutically acceptable carrier and/or excipient.

According to the present invention this object is solved by using a peptide of the present invention or a peptide comprising or consisting of an amino acid sequence of SEQ ID Nos. 14-16 or a peptide-HLA-C monomer complex of the present invention or a peptide-HLA-C multimer complex of the present invention or a pharmaceutical composition of the present invention or a protein comprising an amino acid sequence of SEQ ID NOs. 22 to 31 or 60 to 76

as biomarker for Psoriasis, preferably Psoriasis vulgaris.

According to the present invention this object is solved by providing a peptide of the present invention or a peptide comprising or consisting of an amino acid sequence of SEQ ID Nos. 14-16 or a peptide-HLA-C monomer complex of the present invention or a peptide-HLA-C multimer complex of the present invention or a pharmaceutical composition of the present invention for use in medicine.

According to the present invention this object is solved by providing a peptide of the present invention or a peptide comprising or consisting of an amino acid sequence of SEQ ID Nos. 14-16 or a peptide-HLA-C monomer complex of the present invention or a peptide-HLA-C multimer complex of the present invention or a pharmaceutical composition of the present invention or a protein comprising an amino acid sequence of SEQ ID NOs. 22 to 31 or 60 to

for use in the diagnosis, prevention, monitoring and/or treatment of Psoriasis, preferably Psoriasis vulgaris.

According to the present invention this object is solved by a method for the diagnosis, prevention, monitoring and/or treatment of Psoriasis, preferably Psoriasis vulgaris, comprising the step of

administering a therapeutically effective amount of at least one peptide of the present invention or a peptide comprising or consisting of an amino acid sequence of SEQ ID Nos. 14-16 or a peptide-HLA-C monomer complex of the present invention or a peptide—HLA-C multimer complex of the present invention or a pharmaceutical composition of the present invention to a subject in need thereof.

According to the present invention this object is solved by providing a compound that is directed against HLA-C for use in the prevention, and/or treatment of Psoriasis, preferably Psoriasis vulgaris,

wherein said compound is selected from an anti-HLA-C antibody or fragment thereof (such as Fab, Fv, scFv), a small molecule (inhibitor), a small interfering RNA.

According to the present invention this object is solved by a method for the prevention and/or treatment of Psoriasis, preferably Psoriasis vulgaris, comprising

(a) inhibiting or blocking the interaction of TCR and HLA-C, and/or

(b) suppressing the expression of HLA-C.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the teiminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “8 to 150 amino acids” should be interpreted to include not only the explicitly recited values of 8 to 150, but also include individual values and sub-ranges within the indicated range. Thus, included in this numerical range are individual values such as 8, 9, 10, 11, 12, 13, 14, 15, 16, 17, 18, 19, 20, 21, 22, 23, 24, 25, 26, 27, 28, 29 , . . . 147, 148, 150 and sub-ranges such as from 8 to 100, from 9 to 50, from 9 to 15, from 8 to 00, etc. This same principle applies to ranges reciting only one numerical value, such as “at least 9 amino acids” or “at least 8 amino acids” or “up to 12 amino acids”. Furthermore, such an interpretation should apply regardless of the breadth of the range or the characteristics being described.

Antigenic Peptides

As discussed above, the present invention provides antigenic peptides.

A peptide according to the present invention comprises or consists of an amino acid sequence of the general formula I

X1ₙ-Arg-X2-X3-Y1-X4-Y2-Arg-Z (I)
wherein
X1 is selected from Phe, Arg, Gly, Met, Ala, Ser, Leu or Val;
n is 0 or 1.
X2 is selected from any amino acid;
X3 is selected from any amino acid;
X4 is selected from Thr, Tyr, Val, Cys, Ser, Ala;
Y1 is selected from Arg, Lys, Gln, or Asn;
Y2 is selected from Arg, Leu or Ser; and
Z is selected from Leu, Met, Ile or Val;
   or
wherein
X1 is selected from Phe, Arg, Gly, Met, Ala, Ser, Leu, Val, His, Tyr, Lys or Trp;
n is 0 or 1.
X2 is selected from any amino acid;
X3 is selected from any amino acid;
X4 is selected from Thr, Tyr, Val, Cys, Ser, Ala, Gly or Arg;
Y1 is selected from Arg, Lys, Gln, or Asn;
Y2 is selected from Arg, Leu or Ser; and
Z is selected from Leu, Met, Ile, Val, Thr or Tyr.

Disclaimer:

The peptides of the general fommla I of the present invention do not comprise one of the following amino acid sequences:

SEQ ID NO. 14
SRASRALRL
SEQ ID NO. 15
LRAGRSRRL
SEQ ID NO. 16
RAGRSRRL
SEQ ID NO. 58
MKRERGALSR ASRALRLAPF VYLLLIQTD
SEQ ID NO. 59
AGPRRCRILR VPDGLLSRAL RAGRSRRLAR VRSVSSGNED
EERRAGSESP ARAPSSSPLS SRAPLPERLE AKGTVALGRA
GDALRLAAEY CPGTRRLRLR LLRAESLVGG APGPRAVRCRL

Preferably, the peptides of the present invention have a length of at least 9 amino acids.

In one embodiment, the peptides of the present invention have a length of at least 8 amino acids. In this embodiment, the peptide does not comprise amino acid X1, i.e. n=0.

Preferably, the peptides of the present invention have a length of at least 8 or 9 amino acids,

preferably about 8 to 150 amino acids or about 9 to 150 amino acids.

more preferably about 8 to 100 amino acids or about 9 to 100 amino acids,

more preferably about 8 to 50 or about 9 to 50 amino acids,

more preferably about 8 to 20 or about 9 to 20 amino acids,

even more preferably about 8 to 15 or about 9 to 15 amino acids, such as up to 12 or 13 amino acids;

In some embodiments, the peptide can have a length of up to the lengths of the amino acid sequence of their parent protein (i.e. the protein they are derived from) shown in SEQ ID Nos. 22 to 31 and 60 to 76, as explained below.

The amino acids of the peptides of the present invention can be L-amino acids or D-amino acids; peptides of the present invention comprise amino acids that are either L-amino acids or D-amino acids.

In one embodiment, a peptide of the present invention comprises further component(s), which is/are preferably selected from
N- and/or C-terminal modification(s)
   such as comprising acetylation and/or amidation of the N- and/or C-teiminus, label(s),
   such as fluorescent dye(s), radioisotope(s) and/or contrast agent(s);
tag(s);
drug(s) or their respective prodrug(s);
carrier or depot(s) for drug(s), prodrug(s) or label(s);
immunogenic epitope(s);
cytotoxin(s),
   such as saporin, gelonin or ricin;
radionuclides,
   such as actinium-225, astatine-211, bismuth-213, iodide-131, optionally with a chelate group
and/or combinations thereof.

The further component(s) is/are preferably covalently attached, such as via an amino acid side chain, a linker, spacer and/or anchor group(s), e.g. a cleavable linker.

Preferably, the peptide of the present invention comprises or is an antigen causing a T cell triggered immune response, preferably a CD8⁺ T cell antigen.

In one embodiment, the peptide of the present invention comprises or is an autoantigen causing a T cell triggered immune response, preferably a CD8⁺ T cell autoantigen.

An “autoantigen” or “autoantigenic peptide” or “self-antigen” as used herein refers to an endogenous tissue constituent that stimulates production of autoantibodies or self-reactive T lymphocytes in the subject in which it occurs and thus stimulates a humoral or cell-mediated immune response against self.

Preferably, the peptides of the present invention are peptide ligands for the T-cell receptor Vα3S1/Vβ13S1.

In a preferred embodiment, the peptide of the present invention is a peptide, wherein
X2 is selected from Ser, His, Cys, Trp, Asn, Ala, Tyr, Gln, Phe, Thr, or Pro;
   such as
   selected from Ser, His, Cys, Trp, Asn, Ala, or Tyr; or
   selected from His, Gln, Ser, Ala or Phe; or
   selected from Thr, Ser, Ala, Trp, Asn, His, Pro or Tyr;

and/or
X3 is selected from Tyr, Arg, Trp, Ser, Ala, His, Phe, Val, Gly, Cys or Glu;
   such as
   selected from Tyr, Arg, Trp, Ser, Ala, His, Phe, or Ala; or
   selected from Val, Gly, Arg, Ser or Cys; or
   selected from Arg, Tyr, Ser, Phe, His, Glu, Gly or Ala;

In a preferred embodiment, the peptide of the present invention is a peptide, wherein
X1, if present, is selected from Phe, Arg, Gly, Met, Ala, Ser, Leu, Val, His, Tyr, Lys or Trp;
   such as
   selected from Phe, Arg, Gly, or Met; or
   selected from Ala, Arg, Ser or Leu; or
   selected from His, Arg, Phe, Tyr, Gly, Lys, Ala, Met or Trp;

and/or
X4 is selected from Thr, Tyr, Val, Cys, Ser, Ala, Gly or Arg
   such as
   selected from Thr, Tyr, Val, Cys or Gly; or
   selected from Ser, Ala, Cys or Thr; or
   selected from Thr, Val, Ser, Tyr, Ala or Arg;

and/or
Y2 is selected from Arg, Leu or Ser; and
   such as
   selected from Arg, or Leu; or
   selected from Arg, Leu or Ser;

and/or
Z is selected from Leu, Met, Ile, Val, Thr or Tyr.

In a preferred embodiment, the peptide of the present invention is a peptide, wherein
Z is Leu; or
Y2 is Arg; or
Y2 is Arg and Z is Leu, Met, Ile or Val; or
Y2 is Arg and Z is Leu;

preferably comprising an amino acid sequence selected from the following formulas

X1-Arg-X2-X3-Y1-X4-Y2-Arg-Leu (Ia)

X1-Arg-X2-X3-Y1-X4-Arg-Arg-Z (Ib)

X1-Arg-X2-X3-Y1-X4-Arg-Arg-Leu (Ia)

wherein

X1, X2, X3, X4 Y1 and are as herein above;

Y2 is selected from Arg, or Leu;

Z is selected from Z is Leu, Met, Ile or Val, preferably Leu or Met.

In one embodiment, the peptides of the present invention are mimotopes.

A “mimotope” as used herein refers to an artificial peptide that acts as a TCR ligand and thus mimics a natural peptide epitope from proteins, carbohydrates or glycolipids.

Preferably, a peptide of the present invention comprises or consists of an amino acid sequence selected from SEQ ID NOs. 1 to 9 and 32.

| SEQ ID NO. | |
|---|---|
| 1 | FRSYRTRRL |
| 2 | FRHRRTLRL |
| 3 | FRCRQYRRL |
| 4 | FRWYRVRRL |
| 5 | RRNWNYRRL |
| 6 | GRASRTRRL |
| 7 | GRNARVRRL |
| 8 | MRSHRYLRL |
| 9 | RRYFRCLRM |
| 32 | VRCWRGRRL |

In one embodiment, the peptides of the present invention are natural ligands of proteins.

Preferably, a peptide of the present invention comprises or consists of an amino acid sequence selected from SEQ ID NOs. 10 to 13 and 17 to 19.

| SEQ ID NO. | | |
|---|---|---|
| 10 | ARHRRSLRL | THEM6 |
| 11 | RRQRRSRRL | RASSF10 |
| 12 | RQRRSRRL | RASSF10 |
| 13 | ARSVRTRRL | ASH1L |
| 17 | VRSRRCLRL | ADAMTL5 |
| 18 | RSRRCLRL | ADAMTL5 |
| 19 | RRFCRTSRM | IL24delE3 |
| 20 | HRHSRILRL | HSPC088 |
| 21 | | SRAVRALRL |

For further details, see also Table 2 below.

In one embodiment, the peptides of the present invention are mutated peptide ligands of the protein ADAMTL5.

Preferably, a peptide of the present invention comprises or consists of an amino acid sequence selected from SEQ ID NOs. 33 to 40.

| SEQ ID NO. | |
|---|---|
| 33 | VRSRRCLRM |
| 34 | VRSRRCLRT |
| 35 | VRSRRCLRV |
| 36 | VRSRRCLRY |
| 37 | ARSRRCLRM |
| 38 | VRARRCLRT |
| 39 | VRSARCLRV |
| 40 | VRSRRALRY |

For further details, see also Table 3 below.

In one embodiment, the peptides of the present invention are microbial peptide antigens.

Preferably, a peptide of the present invention comprises or consists of an amino acid sequence selected from SEQ ID NOs. 41 to 51.

| SEQ | | |
|---|---|---|
| ID NO. | Source | |
| 41 | HRTRRTRRL | Serpula lacrymans var. lacrymans |
| | | S7.9 |
| 42 | RRTRRTRRL | Mycobacterium tuberculosis |
| 43 | FRSYRVRRL | Chlamydia trachomatis |
| 44 | GRASRSRRL | Aspergillus niger |
| 45 | KRSRRVLRL | Klebsiella pneumoniae |
| 46 | RRTRRSRRL | Nocardia brasiliensis |
| 47 | MRWFRVRRL | Pseudomonas syringae |
| 48 | WRHSRALRL | Pectobacterium atrosepticum |
| 49 | GRTSRTRRL | Burkholderia lata |
| 50 | RASRTRRL | Desulfovibrio aminophilus |
| 51 | RAARTRRL | Flavonifractor plautii |

For further details, see also Table 4 below.

In one embodiment, the peptides of the present invention are food antigens.

Preferably, a peptide of the present invention comprises or consists of an amino acid sequence selected from SEQ ID NOs. 52 to 57.

| SEQ | | |
|---|---|---|
| ID NO. | Source | |
| 52 | YRSYRTRRM | Coffea canephora |
| 53 | ARSFRSLRL | Malus domestica: Golden Delicious |
| 54 | ARSSRSLRL | Malus domestica: Golden Delicious |
| 55 | HRNHRYLRL | Spinacia oleracea |
| 56 | RRPERRRRL | Triticum aestivum |
| 57 | RRPGRRRRL | Triticum aestivum |

For further details, see also Table 5 below.

Peptide-HLA-C Complexes

As discussed above, the present invention provides complexes of the antigenic peptides of the present invention with HLA-C.

HLA-C belongs to the MHC (human=HLA) class I heavy chain receptors. The C receptor is a heterodimer consisting of a HLA-C mature gene product and β2-microglobulin. The mature C chain is anchored in the cell membrane. MHC Class I molecules, like HLA-C, are expressed by all nucleated cells, and present small peptides to the immune system which surveys for peptide antigens derived from autologous or non-self proteins.

The complexes of the present invention can, for example, be obtained in vitro from a recombinant HLA-C which folds back in presence of the peptide of the present invention.

A peptide-HLA-C monomer complex according to the present invention comprises

(a) at least one peptide selected from a peptide of the present invention or a peptide comprising or consisting of an amino acid sequence of SEQ ID Nos. 14-16,

(b) a HLA-C monomer binding to T-cell receptor(s) (TCR(s)).

The HLA-C is preferably HLA-C*06:02.

A peptide-HLA-C multimer complex according to the present invention comprises a HLA-C multimer which comprises at least two HLA-C monomers defined as (b) herein and which each comprises at least one peptide of the present invention.

In one embodiment, the HLA-C comprises of the monomer or multimer complexes of the present invention comprises further component(s), such as
label(s),
   such as fluorescent dye(s), radioisotope(s) and/or contrast agent(s);
tag(s);
drug(s) or their respective prodrug(s);
carrier or depot(s) for drug(s), prodrug(s) or label(s);
immunogenic epitope(s);
cytotoxin(s),
   such as saporin, gelonin or ricin;
radionuclides,
   such as actinium-225, astatine-211, bismuth-213, iodide-131, optionally with a
chelate group
coupling moiety/moieties;
and/or combinations thereof

The further component(s) is/are preferably covalently attached, such as via an amino acid side chain, a linker, spacer and/or anchor group(s),
e.g. a cleavable linker.

Preferably, the coupling moiety/moieties are comprised in the HLA-C monomer and are suitable for obtaining HLA-C oligomers or multimers.

Examples for such coupling moiety/moieties are

(1) Dextrane

Usually 10 to 25 HLA-C monomers covalently attach to one dextrane molecule (and form a so-called dextramer). See e.g. Scholler et al., 2010.

(2) Biotin

The multimerization can e.g. be obtained via the well-known interaction of biotin with streptavidin or avidin

In one embodiment, the HLA-C comprises biotin as coupling moiety which then interacts with streptavidin or avidin that can e.g. be attached to a surface, such as in a microtiter plate/well or the like.

For example, the most commonly used MHC or HLA-C multimers are tetramers. These are typically produced by biotinylating soluble MHC or HLA-C monomers, which are typically produced recombinantly in eukaryotic or bacterial cells. These monomers then bind to a backbone, such as streptavidin or avidin, creating a tetravalent structure. These backbones can be further conjugated with fluorochromes and can be used to subsequently isolate bound T-cells via flow cytometry.

See e.g. Altmann et al., 1996.

(3) IgG-Fc regions

The HLA-C chain can be linked to the IgG-Fc region of immunoglobulins, creating HLA-C-IgG-Fc fusion proteins.

Preferred HLA-C multimers in the peptide-HLA-C multimer complexes of the present invention are comprised of 2, 3, 4, 5, 6, 7, 8, 9, 10 or more HLA-C monomers, such as dimers, trimmers, tetramers, pentamers, octamers, and the like.

In one embodiment, oligomers with a distribution of monomers per aggregates are comprised in the peptide-HLA-C multimer complexes of the present invention. For example, in case of “dextramers”: usually 10 or 12 to 25 peptide-HLA-C monomers covalently attach to one dextrane molecule. The distribution width and/or maximum can depend on the type of synthesis.

In one embodiment, the multimer complex of the present invention is a HLA-C*06:02 multimer, more preferably a HLA-C*06:02 tetramer.

In one embodiment, the multimer complex of the present invention is a HLA-C*06:02 multimer, more preferably a HLA-C*06:02 dextramer,

preferably comprising 10 to 25 HLA-C monomers,

or preferably comprising 2 to 10 HLA-C monomers.

In one embodiment, the oligomerization or multimerization can be obtained by surface coating and/or the use of polymers. For example, ELISA plates or microtiter plates or Elisaspot plates can be surface-coated with peptide-HLA-C complexes by suitably coating the surfaces with dextrane or streptavidin/avidin or the like.

In one embodiment, the at least one peptide (a) of the monomer or multimer complex is altered for modifying the binding affinity of the peptide or the peptide-HLA-C complex to the TCR.

Such “altered” peptide refers to an analogue peptide with altered primary and/or secondary structure, obtained e.g. by one or more amino acid substitutions at any residue in the amino acid sequence of the peptide.

Such alteration or modification can be used to tune or modify the affinity of the peptide-protein complex (peptide/HLA-C) to the (pathogenic) T cell receptor, such as to obtain stronger agonists or antagonists to put the T cells to rest or to induce anergy or apoptosis.

See Tan et al. (2015) which show that TCR binding affinity governs the functional profile of cancer-specific CD8+ T cells.

See Cole et al. (2014) which show that TCR-peptide specificity overrides affinity-enhancing TCR-MHC complex interactions.

Pharmaceutical Compositions

As discussed above, the present invention provides phattitaceutical compositions.

A phattnaceutical composition of the present invention comprises:

(i) at least one peptide of the present invention and/or a HLA-C multimer as defined

herein;

or a peptide-HLA-C complex of the present invention;

(ii) optionally, a pharmaceutically acceptable carrier and/or excipient.

In one embodiment, the pharmaceutical composition comprises two, three, four or more peptides of the present invention.

Uses as Psoriasis Biomarker

As discussed above, the present invention provides the use of
a peptide of the present invention,
a peptide comprising or consisting of an amino acid sequence of SEQ ID Nos. 14-16,
a peptide-HLA-C monomer complex of the present invention,
a peptide-HLA-C multimer complex of the present invention,
a pharmaceutical composition of the present invention, or
a protein comprising or consisting of an amino acid sequence of SEQ ID NOs. 22 to 31
a protein comprising or consisting of an amino acid sequence of SEQ ID NOs. 60 to 76

as biomarker for Psoriasis.

The proteins with amino acid sequence of SEQ ID NOs. 22 to 31 refer to:

ASH1L_HUMAN; Histone-lysine N-methyltransferase ASH1L (EC 2.1.1.43) (ASH1-like protein) (huASH1) (Absent small and homeotic disks protein 1 homolog) (Lysine N-methyltransferase 2H) and isoforms [SEQ ID NO. 22]

ATL_5 HUMAN; ADAMTS-like protein 5 (ADAMTSL-5) (Thrombospondin type-1 domain-containing protein 6) [SEQ ID NO. 23]

HECAM_HUMAN; Hepatocyte cell adhesion molecule (Protein hepaCAM) [SEQ ID NO. 24]

C2C4A_HUMAN und C2C4B_HUMAN; C2 calcium-dependent domain-containing protein 4A/4B; Nuclear-localized factor 1 (Protein FAM148A) und Nuclear-localized factor 2) (Protein FAM148B)
C2C4A_HUMAN [SEQ ID NO. 25]
C2C4B_HUMAN [SEQ ID NO. 26]

THEM6_HUMAN; Protein THEM6 (Mesenchymal stem cell protein DSCD75) (Thioesterase superfamily member 6)
[SEQ ID NO. 27]

RASFA_HUMAN; Ras association domain-containing protein 10 (RASSF10)
[SEQ ID NO. 28]

Q2YHE6_HUMAN; IL-24 splice variant delE3
[SEQ ID NO. 29]

Q9P0E8_HUMAN; HSPC088 [SEQ ID NO. 30]

Hypothetical protein LOC283951, Sequence ID: gb|AAK61280.1|AE006467_6
[SEQ ID NO.31]

See also Table 2 below.

| SEQ ID NO. | | |
|---|---|---|
| 14 | SRASRALRL | Hepacam |
| 15 | LRAGRSRRL | C2CD4A/B |
| 16 | RAGRSRRL | C2CD4A/B |

The proteins with amino acid sequence of SEQ ID NOs. 60 to 76 refer to:

SEQ ID NO. 60: hypothetical protein SERLADRAFT_432168 [Serpula lacrymans var. lacrymans S7.9]

SEQ ID NO. 61: phenolpthiocerol synthesis type-I polyketide synthase PPSA [Mycobacterium tuberculosis]

SEQ ID NO. 62: Uncharacterised protein [Chlamydia trachomatis]

SEQ ID NO. 63: unnamed protein product [Aspergillus niger]

SEQ ID NO. 64: tail protein [Klebsiella pneumoniae]

SEQ ID NO. 65: hypothetical protein NBRGN 050_00490 [Nocardia brasiliensis]

SEQ ID NO. 66: hypothetical protein [Pseudomonas syringae]

SEQ ID NO. 67: CDP-diglyceride synthetase [Pectobacterium atrosepticum]

SEQ ID NO. 68: hypothetical protein [Burkholderia lata]

SEQ ID NO. 69: hypothetical protein [Desulfovibrio aminophilus]

SEQ ID NO. 70: Protein of uncharacterised function (DUF3847) [Flavonifractor plautii or Clostridiales bacterium VE202-03]

SEQ ID NO. 71: unnamed protein product [Coffea canephora]

SEQ ID NO. 72: uncharacterized protein LOC103417966 [Malus domestica]

SEQ ID NO. 73: uncharacterized protein LOC103417966 [Malus domestica]

SEQ ID NO. 74: hypothetical protein SOVF 054900 isoform A [Spinacia oleracea]

SEQ ID NO. 75: abscisic acid-induced protein, partial [Triticum aestivum]

SEQ ID NO. 76: abscisic acid-induced protein, partial [Triticum aestivum]

The use according the present invention comprises at least one of the following:
monitoring disease activity;
monitoring efficacy of treatment;
deteimining disease risk; and/or
deteiinining the frequency of autoreactive T cells in samples of subjects having Psoriasis, preferably Psoriasis vulgaris, more preferably Psoriasis vulgaris with all types of (clinical) manifestations, such as but not limited to chronic plaque psoriasis, acute guttate psoriasis, localized or generalized pustular psoriasis, psoriatic arthritis, psoriatic nail disease and others.

“Psoriasis vulgaris” as used herein refers to Psoriasis vulgaris with all types of (clinical) manifestations, such as but not limited to chronic plaque psoriasis, acute guttate psoriasis, localized or generalized pustular psoriasis, psoriatic arthritis, psoriatic nail disease and others.

Medical Uses

As discussed above, the present invention provides
the peptide of the present invention,
a peptide comprising or consisting of an amino acid sequence of SEQ ID Nos. 14-16,
the peptide-HLA-C monomer complex of the present invention,
the peptide-HLA-C multimer complex of the present invention, or
the phatinaceutical composition of the present invention,

for use in medicine.

As discussed above, the present invention provides
the peptide of the present invention,
a peptide comprising or consisting of an amino acid sequence of SEQ ID Nos. 14-16,
the peptide-HLA-C monomer complex of the present invention,
the peptide-HLA-C multimer complex of the present invention,
the pharmaceutical composition of the present invention,
a protein comprising or consisting of an amino acid sequence of SEQ ID NOs. 22 to 31, or
a protein comprising or consisting of an amino acid sequence of SEQ ID NOs. 60 to 76

for use in the diagnosis, prevention, monitoring and/or treatment of Psoriasis, preferably Psoriasis vulgaris.

In one embodiment comprises the monitoring of Psoriasis, preferably Psoriasis vulgaris, monitoring the treatment or therapy of Psoriasis, preferably Psoriasis vulgaris.

In one embodiment,

determining the frequency of autoreactive T cells in samples of subjects having Psoriasis, preferably Psoriasis vulgaris, is comprised.

One embodiment comprises the use of a peptide of the present invention or a peptide comprising or consisting of an amino acid sequence of SEQ ID Nos. 14-16, or pharmaceutical composition (comprising at least one peptide) of the present invention or protein of the present invention, wherein the prevention and/or treatment of Psoriasis, preferably Psoriasis vulgaris, comprises
inhibiting or blocking the interaction of TCR and HLA-C, and/or
suppressing the expression of HLA-C.

One embodiment comprises the use of a complex of the present invention, wherein the prevention and/or treatment of Psoriasis, preferably Psoriasis vulgaris, comprises
creating immunotolerance against the autoantigens;
affecting, tolerating or blocking the activation of autoreactive T cells for therapeutic purposes;
eliminating the pathogenic CD8⁺ T cells; and/or
down-regulating the pathogenic immune response by specifically blocking HLA-C/T-cell-interactions.

Preferably, the peptide and/or the HLA-C of the complex comprises at least a toxin or radionuclide or fluorescent dye or any other traceable marker, as described for example herein, as further component(s).

Preferably, the administration is systemic, intravenous, and/or via intradermal, subcutaneous, intramuscular, intravenous, intraosseous, intraperitoneal, intrathecal, epidural, intracardiac, intraarticular, intracavernous, intracerebral, intracerebroventricular and intravitreal inj ection(s).

### Methods of Treatment

As discussed above, the present invention provides a method for the diagnosis, prevention, monitoring and/or treatment of Psoriasis, preferably Psoriasis vulgaris.

Said method comprises the step of

administering a therapeutically effective amount of
at least one peptide of the present invention,
at least one peptide comprising or consisting of an amino acid sequence of SEQ ID Nos. 14-16,
a peptide-HLA-C monomer complex of the present invention,
a peptide-HLA-C multimer complex of the present invention,
a pharmaceutical composition of the present invention,
a protein comprising or consisting of an amino acid sequence of SEQ ID NOs. 22 to 31, or
a protein comprising or consisting of an amino acid sequence of SEQ ID NOs. 60 to 76

to a subject in need thereof.

Preferably, the administration is systemic, intravenous, and/or via intradermal, subcutaneous, intramuscular, intravenous, intraosseous, intraperitoneal, intrathecal, epidural, intracardiac, intraarticular, intracavernous, intracerebral, intracerebroventricular and intravitreal inj ection(s).

In one embodiment, the peptide and/or the HLA-C of the complex comprises at least a toxin or radionuclide or fluorescent dye or any other traceable marker, as described for example herein, as further component(s).

Means and Methods for Inhibiting or Blocking the Interaction of TCR and HLA-C As discussed above, the present invention provides a compound that is directed against HLA-C for use in the prevention, and/or treatment of Psoriasis, preferably Psoriasis vulgaris.

The HLA-C is preferably HLA-C*06:02.

Said compound is selected from an anti-HLA-C antibody or fragment thereof (such as Fab, Fv, scFv), a small molecule (inhibitor), a small interfering RNA.

Disclaimer:

The compounds do not comprise an isolated V_{L} domain or an isolated V_{F} domain, in particular in an embodiment where the compounds are fragments of an anti-HLA-C antibody or an anti-HLA-C*06:02 antibody.

Preferably the prevention and/or treatment of Psoriasis, preferably Psoriasis vulgaris, comprises

(a) inhibiting or blocking the interaction of TCR and HLA-C, and/or

(b) suppressing the expression of HLA-C.

The HLA-C is preferably HLA-C*06:02.

As discussed above, the present invention provides a method for the prevention and/or treatment of Psoriasis, preferably Psoriasis vulgaris, comprising

(a) inhibiting or blocking the interaction of TCR and HLA-C, and/or

(b) suppressing the expression of HLA-C.

The HLA-C is preferably HLA-C*06:02.

In one embodiment, the inhibiting or blocking the interaction of TCR and HLA-C (a) comprises: interfering with the contact between TCR and HLA-C
such as but not limited to antibodies (monoclonal antibodies, antibody fragments, e.g. Fab, Fv, scFv) or other molecules directed specifically against HLA-C.

In one embodiment, the suppressing the expression of HLA-C (b) comprises: suppressing the expression of HLA-C on cell surfaces by means
such as but not limited to small interfering RNAs or other small molecules that are designed to reduce HLA-C transcription, HLA-C translation or HLA-C transport to the cell surface.

The HLA-C is preferably HLA-C*06:02.

### FURTHER DESCRIPTION OF PREFERRED EMBODIMENTS

Abstract

Proteins Histone-lysine N-methyltransferase ASH1L, ADAMTS-like protein 5 (ADMTSL5), Ras association domain-containing protein 10 (RASSF10), Hepatocyte cell adhesion molecule (HEPACAM), Protein THEM6, C2 calcium-dependent domain-containing protein 4A/4B (C2CD4A/4B), IL-24 splice variant delE3, HSPC088 and hypothetical protein LOC283951 and/or peptides from said proteins and/or corresponding mimotopes for the diagnosis and treatment of psoriasis either alone or in the complex with HLA-C*06-monomers or multimers.

Blocking the interaction between T-cell receptor (TCR) and Human Leukocyte Antigen (HLA)-C molecules or any mechanism that downregulates HLA-C in skin or in other tissues as a specific treatment for psoriasis vulgaris.

The present invention relates to selected autoantigens, corresponding peptide epitopes and altered peptide ligands, such as corresponding mimotopes, as diagnostic tools or therapeutic agents, compositions comprising them as well as processes for the diagnosis, monitoring and treatment of psoriasis vulgaris.

The present invention also relates to the application of HLA-C-directed approaches that block the interaction with T-cell antigen receptors and thus inhibit T-cell activation for the specific treatment of psoriasis vulgaris in psoriasis patients.

The present invention is based on the discovery that the human leukocyte antigen HLA-C*06:02 directs an autoimmune response against melanocytes and presents autoantigens to pathogenic T cells in psoriasis. Using a recombinant TCR from a pathogenic lesional psoriatic T-cell clone of a HLA-C*06-positive psoriasis patient it identifies epitopes from autoantigenic proteins in psoriasis. It shows that patients have circulating T cells in peripheral blood, which specifically react against complexes composed of these peptides and HLA-C*06, and that complexes of said peptides and HLA-C*06 can be used to identify these peptide-specific T cells. The invention also shows that blocking the interaction between HLA-C*06 and a pathogenic T-cell antigen receptor can inhibit T-cell activation. Blocking the interaction between HLA-C molecules and TCR is therefore a treatment modality in psoriasis.

Psoriasis vulgaris is a frequent HLA-C*06:02-associated T-cell mediated autoimmune skin disease. The invention-related findings are based on the pioneering usage of a human TCR to
determine the functional role of HLA-C*06:02 in presenting autoantigenic peptides,
identify the target cell of the skin-specific autoimmune response,
identify specific antigens of the autoimmune response.

HLA-C*06:02 is the main psoriasis risk allele and present in ⅔ of all patients with psoriasis.

Herein we prove for the first time,
that HLA-C*06:02 acts by directing a T-cell mediated autoimmune response of CD8⁺ T cells against melanocytes,
that HLA-C*06:02 presents autoantigenic peptides to pathogenic CD8⁺ T cells,
identify said antigenic peptides peptides that are presented as autoantigens from melanocytes
show, that blocking the interaction between the HLA-C*06:02/peptide complex with a T-cell receptor from CD8⁺ T cells inhibits antigen-specific immune activation.

The invention can be used for:

(i) Diagnosis of psoriasis:
Use of multimeric peptide/HLA-C*06:02 complexes as biomarker for
   monitoring disease activity,
   monitoring efficacy of treatment,
   defining disease risk.

(ii) Treatment of psoriasis:
Use of altered peptide ligands for creating immunotolerance against the autoantigens,
Provide a cure by eliminating the pathogenic CD8⁺ T cells using toxin-coupled multimeric peptide/HLA-C*06:02 complexes,
Downregulate the pathogenic immune response by specifically blocking HLA-C/T-cell-interactions.
Results

Generation of Vα3S1/Vβ13S1 TCR hybridoma

We had previously developed a technique for the molecular characterization of the paired TCR α and β-chain rearrangements of single T cells. Herewith, we were able to prove that the majority of CD8⁺ T cells, which infiltrate into the epidermis of psoriatic plaques, are clonally expanded (Kim et al., 2012). Clonal T-cell expansions in inflammatory tissue lesions are indicative of antigen-driven T-cell responses and characterize presumably pathogenic T cells (Kent et al., 2005; see also WO 2012/017081 A1).

Now, we cloned the cDNA of the α- and β-chains of a functional Vα3S1/Vβ13S1 TCR from a predominant epidettnal psoriatic CD8⁺ T-cell clone of an HLA-C*06:02-positive psoriasis patient. Evidence of psoriasis-related pathogenicity of the donor T cells is based on three observations:
(1) Multiple dennal and epidermal CD8⁺ T cells from a lesional psoriasis biopsy carried the same TCR α-chain (Vα3S1-NN-Jα 45.1: CA TDAL YSGG) and β-chain rearrangements (Vβ13S1-N(D)N-Jβ1.1: CASSY SEGED EAFF; Arden nomenclature (Arden et al., 1995), deduced amino acid sequence, one letter amino acid code).
(2) The clonal Vα3S1/Vβ13S1 TCR was also identified in a second, independently examined biopsy part from the same patient (Kim et al., 2012).
(3) CD8⁺ T cells in psoriatic epidermis have been described to preferentially rearrange Vβ13S1 (Chang et al., 1997).

We co-expressed this Vα3S1/Vβ13S1 TCR together with human CD8 α and β chains and super green fluorescence protein (sGFP) under the control of nuclear factor of activated T cells (NFAT) in the TCR-deficient mouse reporter T-hybridoma cell line 58α⁻β⁻ (Seitz et al., 2006). Upon TCR ligation, Vα3S1/Vβ13S1 TCR hybridoma cells are activated and produce sGFP, which can be detected by ultraviolet (UV) fluorescence microscopy or fluorescence activated cell sorting (FIG. 1). When assessing the results given below, however, it needs to be considered that the percentage of activated i.e. green-fluorescing cells in this complex cell-based recombinant assay may differ between different experiments. It may be influenced by the stability of the respective hybridoma cell batch and, in co-culture experiments, by the quantitative ratio of stimulatory and hybridoma cells, which decides the probability of cell-cell contacts (Siewert et al., 2011).

The Vα3S1/Vβ13S1 l reacts against melanocytes in an HLA-C*06:02 restricted manner Given that the Vα3S 1/Vβ13S1-TCR hybridoma carries the antigen-specificity of pathogenic psoriatic CD8⁺ T cells, we used it to determine HLA-class I restriction and antigen-specificity of the lesional psoriatic immune response. The TCR origin from a lesional epidermal CD8⁺ T-cell clone of an HLA-C*06:02-positive psoriasis patient indicated an HLA-C*06:02-restricted autoreactivity against skin-resident tissue cells. Therefore, we assessed the effect of various skin-derived cell types on the activation of the Vα3S 1/Vβ13S1-TCR hybridoma in co-culture experiments.

We isolated primary melanocytes from the skin and seeded them in explant culture. Cell type was confirmed by the contents of brownish melanosomes and staining with a p75-specific antibody against melanocytes. The Vα3S 1/Vβ13S1-TCR hybridoma cells were activated upon co-culture with primary human melanocytes derived from the skin of HLA-C*06:02-positive but not HLA-C*06:02-negative donors, as evidenced by the induction of sGFP (FIGS. 2A and 2B). This effect was further enhanced, when HLA-C*06:02-positive melanocytes were preincubated with interferon (IFN)-γ, which increases the expression of HLA-class I molecules (FIG. 2C). The stimulatory capacity was observed in HLA-C*06:02-positive melanocytes obtained from both skin of psoriasis patients or healthy donors, indicating a genuine antigenicity of melanocytes in the context of HLA-C*06:02. TCR-hybridoma activation was HLA-restricted. It was increased by the addition of IFN-γ, which enhances the expression of HLA-C, and completely blocked by a pan HLA-class I antibody, W6/32, that inhibits TCR-HLA interactions (FIG. 2B).

Consistent results were obtained with two inherently HLA-C*06:02-positive melanocytic cell lines, 1205LU and WM278: cells from both cell lines strongly activated the TCR hybridoma when preincubated with IFN-y to induce the expression of HLA-C (FIG. 2D). Again, this effect was reverted by a blocking pan-HLA-class I antibody, W6/32. The HLA-C*06:02-negative melanocytic cell lines, WM9, WM1232 or WM239, activated Vα3S1/Vβ13S1 TCR hybridoma cells in co-culture experiments only after transfection of HLA-C*06:02, but not HLA-A*0201 (FIG. 2E). The stimulatory capacity of these HLA-C*06:02-transfected cell lines was independent from addition of IFN-γ.

No hybridoma activation was observed in co-culture experiments with HLA-C*06:02-transfected primary human keratinocytes, cells from the epidettnal cell line, HaCaT (Boukamp et al., 1988) or A431, nor with HLA-C*06:02-positive primary skin fibroblasts (data not shown), indicating a specific role of melanocytes in TCR activation. Thus, our results imply a selective reactivity of the psoriatic Vα3S 1/Vβ13S1-TCR of a presumably pathogenic T-cell clone against self-antigens presented by melanocytes in dependence of HLA-C*06:02-surface expression.
HLA-C directs an autoimmune response against melanocytes in psoriasis. Increasing the expression of HLA-C enhances TCR-mediated T-cell activation. Vice versa, blocking the interaction between HLA-C and TCR blocks TCR-mediated T-cell activation. Blocking the interaction between HLA-C and TCR is a relevant approach for the treatment of psoriasis.
Given the role of HLA-C in presenting the autoantigens any mechanism that downregulates the expression of HLA-C in skin lesions or other tissues can be used to control the autoimmune response and be employed for treatment of psoriasis.

The Vα3S1/Vβ13S1 TCR reacts against different peptides with a convergent amino acid pattern that facilitates the identification of stimulatory natural human peptide homologues To characterize potential melanocytic self-antigens, we searched for peptide motifs that are recognized by the Vα3S1/Vβ13S1 TCR in the context of HLA-C*06:02. For this purpose, we used a randomized plasmid peptide library, encoding nonameric peptides (Siewert et al., 2011). In previous experiments we had shown that this approach is suitable for identifying HLA-restricted peptide antigens of a TCR specific for a defined influenza virus antigen. COS-7 cells were cotransfected with the peptide library and pRSV-HLA-C*06:02 and incubated with Vα3S 1/Vβ13S1-TCR hybridoma cells. COS-7 cells adjacent to activated TCR hybridoma cells were picked and subjected to plasmid subcloning and sequencing to identify the encoded antigenic peptide as described (Siewert et al., 2011).

We identified nine different nonamer mimotopes, i.e. artificial peptide epitopes that ligated the Vα3S1/Vβ13S1 TCR in the context of HLA-C*06:02 (Table 1, FIGS. 4 and 5).

TABLE 1
Amino acid sequence of mimotopes activation
Vα3S1/Vβ13S1 TCR hybridoma cells

| Mimotope | | | | | | | | | |
|---|---|---|---|---|---|---|---|---|---|
| SEQ ID | Amino acid positions P1-P9 | | | | | | | | |
| NO. | [symbol] | P2 | [symbol] | [symbol] | P5 | [symbol] | P7 | P8 | P9 |
| 1 | F | R | S | Y | R | T | R | R | L |
| 2 | F | R | H | R | R | T | L | R | L |
| 3 | F | R | C | R | Q | Y | R | R | L |
| 4 | F | R | W | Y | R | V | R | R | L |
| 5 | R | R | N | W | N | Y | R | R | L |
| 6 | G | R | A | S | R | T | R | R | L |
| 7 | G | R | N | A | R | V | R | R | L |
| 8 | M | R | S | H | R | Y | L | R | L |
| 9 | R | R | Y | F | R | C | L | R | M |
| 32 | V | R | C | W | R | G | R | R | L |

Amino acids are given as single letter amino acid code
Bold: conserved amino acid pattern in positions P2, P5, P8 and P9; italic: diversity in positions P1, P3, P4 and P6.

These nonamers exhibited diversity in positions P1, P3, P4 and P6. However, our analysis revealed a conserved amino acid pattern in positions P2 and P8 (Arg), P9 (Leu or Met) and P5 (Arg, Glu or Asn), indicating a high relevance of these amino acids for the interaction with the Vα3S1/Vβ13S1 TCR. The observed polyspecificity of the Vα3S1/Vβ13S1 TCR is consistent with the recognition behaviour of TCRs reported previously (Wucherpfennig et al., 2007). Defined TCRs may cross-react with many distinct peptides, which, however, share conserved TCR-binding motifs that can be used to identify the naturally occurring TCR ligands ((Siewert et al., 2011; Birnbaum et al., 2014).

To identify the corresponding natural psoriatic antigen of the Vα3S1/Vβ13S1 TCR, we screened the mimotopes against the non-redundant human protein database and the transcriptome of 1205LU cells. However, we were unable to identify proteins containing peptides that were identical to any of the mimotopes in this way. For further homology searches, we therefore generated various algorithms based on the converging amino acid pattern of the above mimotopes. This allowed us to select 185 different peptides from natural human proteins for further analysis.

After cloning into an expression vector they were co-transfected with HLA-C*06:02 into COS-7 cells and tested for their ability to activate Vα3S1/Vβ13S1 TCR hybridoma cells in co-culture experiments. Nine of these peptide epitopes stimulated the Vα3S1/Vβ13S1 TCR hybridoma (Table 2; FIG. 6: Antigen-specific activation of Vα3S1/Vβ13S1 TCR hybridoma by select peptides). Thus, analysis of the convergent amino acids pattern of library mimotopes allowed for the identification of several putative self-epitopes presented by HLA-C*06:02 and recognized by the Vα3S1/Vβ13S1 TCR.

TABLE 2
Peptides from natural human proteins that
activate the Vα3S1/Vβ13S1 TCR hybridoma

| | UniProte | Uniprot | | | Peptide |
|---|---|---|---|---|---|
| # | Accession # | entry | Protein name | Gen name | sequence |
| 1 | Q9NR48 | ASH1L_ | Histone-lysine N- | ASH1L, | ARSVRTRRL |
| | | HUMAN | methyltransferase | KIAA1420, | SEQ ID NO. 13 |
| | | | ASH1L (EC 2.1.1.43) | KMT2H | |
| | | | (ASH1-like protein) | | |
| | | | (huASH1) (Absent small | | |
| | | | and homeotic disks protein | | |
| | | | 1 homolog) (Lysine N- | | |
| | | | methyltransferase 2H) and | | |
| | | | isoforms | | |
| 2 | Q6ZMM2 | ATL5_ | ADAMTS-like protein 5 | ADAMTSL5, | VRSRRCLRL |
| | | HUMAN | (ADAMTSL-5) | THSD6 | SEQ ID NO. 17 |
| | | | (Thrombospondin-5) | | |
| | | | domain-containing protein 6) | | |
| 3 | Q14CZ8 | HECAM_ | Hepatocyte cell adhesion | HEPACAM | SRASRALRL |
| | | HUMAN | molecule (Protein hepaCAM) | | SEQ ID NO. 14 |
| 4 | Q8NCU7 | C2C4A_ | C2 calcium-dependent | C2CD4A/B, | LRAGRSRRL |
| | A6NLJ0 | HUMAN | domain-containing protein | FAM148B, | SEQ ID NO. 15 |
| | | C2C4B_ | 4A/4B Nuclear-localized | NLF2 | |
| | | HUMAN | factor 1 (Protein FAM148A) | | |
| | | | Nuclear-localized factor 2) | | |
| | | | (Protein FAM148B) | | |
| 5 | Q8WUY1 | THEM6_ | Protein THEM6 | THEM6, | ARHRRSLRL |
| | | HUMAN | (Mesenchymal stem cell | C8orf55, | SEQ ID NO. 10 |
| | | | protein DSCD75) | PSEC0098 | |
| | | | (Thioesterase superfamily | | |
| | | | member 6) | | |
| 6 | A6NK89 | RASFA_ | Ras association domain- | RASSF10 | RRQRRSRRL |
| | | HUMAN | containing protein 10 | | SEQ ID NO. 11 |
| 7 | Q2YHE6 | Q2YHE6_ | IL-24 splice variant delE3 | N/A | RRFCRTSRM |
| | | HUMAN | | | SEQ ID NO. 19 |
| 8 | Q9P08E8 | Q9P0E8_ | HSPC088 | | HRHSRILRL |
| | | HUMAN | | | SEQ ID NO. 20 |
| 9 | | GenBank: | hypothetical protein | | SRAVRALRL |
| | | AAK61280.1 | LOC283951, isoform CRA_a | | SEQ ID NO. 21 |

Peptides from several natural human proteins constitute autoantigens for psoriasis when complexed with HLA-C*06:02 and activate a representative pathogenic psoriatic TCR. Mimotopes (artificial TCR peptide ligands) sharing a particular amino acid pattern can be used to replace these natural peptide ligands for TCR activation. Due to the polyspecificity/crossreactivity of TCRs, a single TCR may recognize all these different natural peptides. Both mimotopes and natural peptide ligands can therefore be used to address pathogenic psoriatic T cells in psoriasis in the context of HLA-C*06 for diagnostic or therapeutic purposes.

Staining of PBMC from Psoriasis Patients with HLA-C*06: 02-Peptide Dextramers

We used FITC-labelled HLA-C*06:02-peptide dextramers to stain CD8⁺ T cells from psoriasis patients. Only HLA-C*06:02-dextramers containing antigenic peptides for the Vα3S1/Vβ13S1 TCR stained CD8⁺ T cells. As negative control we used an unrelated peptide, VRHDGGNVL (Falk et al., 1993), complexed with HLA-C*06:02-dextramers did not bind to blood T cells from psoriasis patients (FIG. 7). Thus, the peptides can be used in complex with HLA-C dextramers to address autoreactive T cells in blood of psoriasis patients.
Autoantigenic peptides complexed with HLA-C*06:02-multimers can be used to identify autoreactive CD8⁺ T cells in psoriasis. This mechanism can be used as biomarker to measure the frequency of autoreactive T cells from biologic samples (blood, skin, lymph nodes etc) in order to support the diagnosis of psoriasis or to monitor the course of disease or assess the risk for disease onset in individuals with a genetic predisposition for psoriasis.
Autoantigenic peptides complexed with toxin-conjugated HLA-C*06:02-dextramers can be used for an antigen-specific elimination of autoreactive pathogenic CD8⁺ T cells in psoriasis.
Altered peptide ligands complexed with HLA-C*06:02-dextramers that bind the pathogenic TCRs can be used to affect or tolerize or block the activation of autoreactive T cells for therapeutic purposes.
Peptides alone or together can be used to measure the T-cell responsiveness in stimulation assays in order to detetrnine specific immune responses in patients.

TABLE 3
Mutated ADAMTSL5 peptide ligands for the
Vα3S1/Vβ13S1 T-cell receptor

| Mutated | | | | | | | | | |
|---|---|---|---|---|---|---|---|---|---|
| ADAMTSL5 | | | | | | | | | |
| Peptide | Amino acid positions P1-P9 | | | | | | | | |
| SEQ ID NO. | P1 | P2 | P3 | P4 | P5 | P6 | P7 | P8 | P9 |
| 33 | V | R | S | R | R | C | L | R | M |
| 34 | V | R | S | R | R | C | L | R | [symbol] |
| 35 | V | R | S | R | R | C | L | R | [symbol] |
| 36 | V | R | S | R | R | C | L | R | [symbol] |
| 37 | [symbol] | R | S | R | R | C | L | R | M |
| 38 | V | R | [symbol] | R | R | C | L | R | T |
| 39 | V | R | S | [symbol] | R | C | L | R | V |
| 40 | V | R | S | R | R | [symbol] | L | R | Y |

Exchange of the amino acids of the ADAMTSL5 nonamer peptide at each position against Ala or of the amino acid at position 2 and 9 against the amino acids Ala, Gly, Thr, Pro or Tyr (P2) or against Ala, Gly, Met, Thr, Val or Tyr (P9) identified mutated ADAMTSL5 peptide ligands of the Vα3S1/Vβ13S1 TCR and defined those amino acid residues that were recognized/required for recognition by the Vα3S1/Vβ13S1 TCR. The Table shows all mutants that activated the Vα3S1/Vβ13S1 TCR.

TABLE 4
Microbial peptide antigens of the Vα3S1/Vβ13S1 T-cell receptor

| | | | | | | | | | | Protein |
|---|---|---|---|---|---|---|---|---|---|---|
| Amino acid positions P1-P9 | | | | | | | | | From | [SEQ ID NO. protein] |
| P1 | P2 | P3 | P4 | P5 | P6 | P7 | P8 | P9 | Genus/Species | [SEQ ID NO. of peptide] |
| H | R | T | R | R | T | R | R | L | Serpula lacrymans | Nonamer: hypothetical |
| | | | | | | | | | var. lacrymans S7.9 | protein |
| | | | | | | | | | | SERLADRAFT_432168 |
| | | | | | | | | | | [SEQ ID NO. 60] |
| | | | | | | | | | fungus | [SEQ ID NO. 41] |
| R | R | T | R | R | T | R | R | L | Mycobacterium tuberculosis | Nonamer: phenolpthiocerol |
| | | | | | | | | | | synthesis type-I polyketide |
| | | | | | | | | | | synthase PPSA |
| | | | | | | | | | | [SEQ ID NO. 61] |
| | | | | | | | | | bacterium | [SEQ ID NO. 42] |
| F | R | S | Y | R | V | R | R | L | Chlamydia trachomatis | Nonamer: Uncharacterised |
| | | | | | | | | | | protein |
| | | | | | | | | | | [SEQ ID NO. 62] |
| | | | | | | | | | bacterium | [SEQ ID NO. 43] |
| G | R | A | S | R | S | R | R | L | Aspergillus niger | Nonamer: unnamed protein |
| | | | | | | | | | | product |
| | | | | | | | | | | [SEQ ID NO. 63] |
| | | | | | | | | | fungus | [SEQ ID NO. 44] |
| K | R | S | R | R | V | L | R | L | Klebsiella pneumoniae | Nonamer: tail protein |
| | | | | | | | | | | [SEQ ID NO. 64] |
| | | | | | | | | | Bakterium | [SEQ ID NO. 45] |
| R | R | T | R | R | S | R | R | L | Nocardia brasiliensis | Nonamer: hypothetical |
| | | | | | | | | | | protein |
| | | | | | | | | | | NBRGN_050_00490 |
| | | | | | | | | | | [SEQ ID NO. 65] |
| | | | | | | | | | bacterium | [SEQ ID NO. 46] |
| M | R | W | F | R | V | R | R | L | Pseudomonas syringae | Nonamer: hypothetical |
| | | | | | | | | | | protein |
| | | | | | | | | | | [SEQ ID NO. 66] |
| | | | | | | | | | bacterium | [SEQ ID NO. 47] |
| W | R | H | S | R | A | L | R | L | Pectobacterium atrosepticum | Nonamer: CDP-diglyceride |
| | | | | | | | | | | synthetase |
| | | | | | | | | | | [SEQ ID NO. 67] |
| | | | | | | | | | bacterium | [SEQ ID NO. 48] |
| G | R | T | S | R | T | R | R | L | Burkholderia lata | Nonamer: hypothetical |
| | | | | | | | | | | protein |
| | | | | | | | | | | [SEQ ID NO. 68] |
| | | | | | | | | | bacterium | [SEQ ID NO. 49] |
| | R | A | S | R | T | R | R | L | Desulfovibrio aminophilus | Octamer: hypothetical |
| | | | | | | | | | | protein |
| | | | | | | | | | | [SEQ ID NO. 69] |
| | | | | | | | | | bacterium | [SEQ ID NO. 50] |
| | R | A | A | R | T | R | R | L | Flavonifractor plautii | Octamer: hypothetical |
| | | | | | | | | | | protein - Octamer of AG 7 |
| | | | | | | | | | | [SEQ ID NO. 70] |
| | | | | | | | | | bacterium | [SEQ ID NO. 51] |

Peptides from pathogens were identified by searching the sequences of the mimotopes and of the human peptides ligating the Vα3S1/Vβ13S1 TCR against protein sequences specified according to bacterium sp. (taxid:77133), viruses (taxid:10239), fungi (taxid:4751) using the Protein BLAST search tool. Candidate peptides were cloned and tested for their ability to activate the Vα3S1/Vβ13S1 in the context of HLA-C*06:02. The table shows those peptides that were recognized by the Vα3S1/Vβ13S1 TCR.

TABLE 5
Food antigens of the Vα3S1/Vβ13S1 T-cell receptor

| | | | | | | | | | | Protein |
|---|---|---|---|---|---|---|---|---|---|---|
| Amino acid positions P1-P9 | | | | | | | | | From | [SEQ ID NO. protein] |
| P1 | P2 | P3 | P4 | P5 | P6 | P7 | P8 | P9 | Genus/Species | [SEQ ID NO. of peptide] |
| Y | R | S | Y | R | T | R | R | M | Coffea canephora | Nonamer: unnamed protein |
| | | | | | | | | | | product |
| | | | | | | | | | | [SEQ ID NO. 71] |
| | | | | | | | | | plant | [SEQ ID NO. 52] |
| A | R | S | F | R | S | L | R | L | Malus domestica: | Nonamer: PREDICTED: |
| | | | | | | | | | Golden Delicious | uncharacterized protein |
| | | | | | | | | | | LOC103417966 |
| | | | | | | | | | | [SEQ ID NO. 72] |
| | | | | | | | | | plant | [SEQ ID NO. 53] |
| A | R | S | S | R | S | L | R | L | Malus domestica: | Nonamer: PREDICTED: |
| | | | | | | | | | Golden Delicious | uncharacterized protein |
| | | | | | | | | | | LOC103417966 |
| | | | | | | | | | | [SEQ ID NO. 73] |
| | | | | | | | | | plant | [SEQ ID NO. 54] |
| H | R | N | H | R | Y | L | R | L | Spinacia oleracea | Nonamer: hypothetical |
| | | | | | | | | | | protein SOVF_054900 |
| | | | | | | | | | | isoform A |
| | | | | | | | | | | [SEQ ID NO. 74] |
| | | | | | | | | | plant | [SEQ ID NO. 55] |
| R | R | P | E | R | R | R | R | L | Triticum aestivum | Nonamer: abscisic acid- |
| | | | | | | | | | | induced protein, partial |
| | | | | | | | | | | [SEQ ID NO. 75] |
| | | | | | | | | | plant | [SEQ ID NO. 56] |
| R | R | P | G | R | R | R | R | L | Triticum aestivum | Nonamer: abscisic acid- |
| | | | | | | | | | | induced protein, partial |
| | | | | | | | | | | [SEQ ID NO. 76] |
| | | | | | | | | | plant | [SEQ ID NO. 57] |

Peptides from food components were identified by searching the sequences of the mimotopes and of the human peptides ligating the Vα3S1/Vβ13S1 TCR protein against all known protein sequences using the Protein BLAST search tool. Candidate peptides were cloned and tested for their ability to activate the Vα3S1/Vβ13S1 in the context of HLA-C*06:02. The table shows those peptides that were recognized by the Vα3S1/Vβ13S1 TCR.

The following examples and drawings illustrate the present invention without, however, limiting the same thereto.

### BRIEF DESCRIPTION OF THE DRAWINGS

FIG. 1. Vα3S1/Vβ13S1-TCR hybridoma activation by CD3-antibody.

Shown is activation of hybridoma cells by a CD3 monoclonal antobody, that leads to the production of green fluorescent protein. This can be visualized by fluorescence microscopy or determined by FACS analysis.

FIG. 2. HLA-C*06:02-Restricted Activation of the CD8⁺ Vα3S1/Vβ13S1 TCR Reporter Hybridoma Cell Line by Melanocytes.

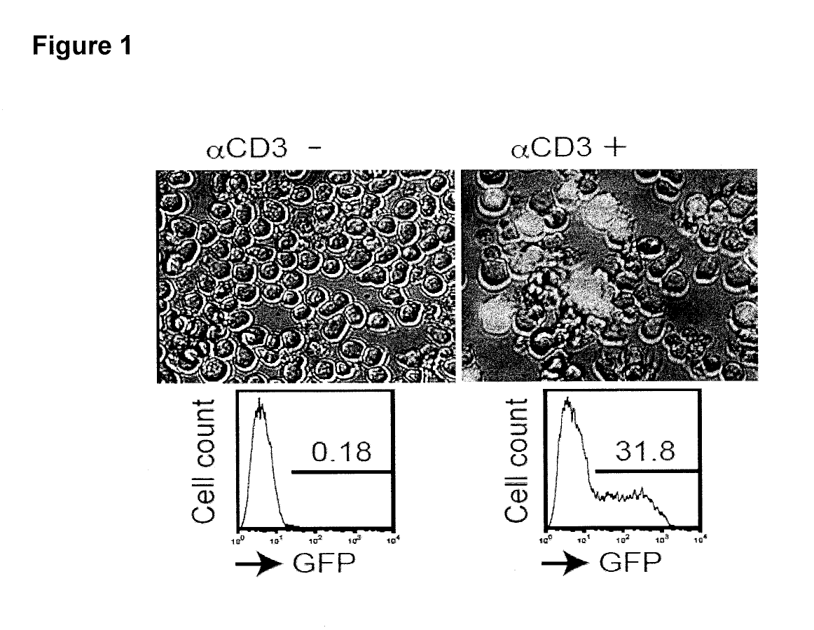

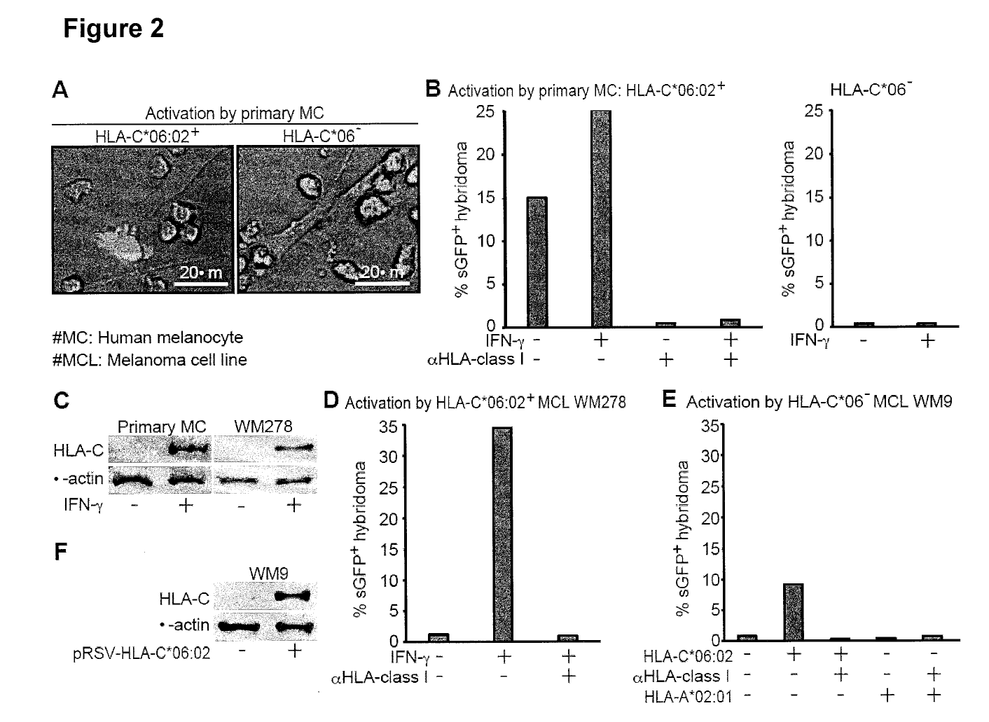

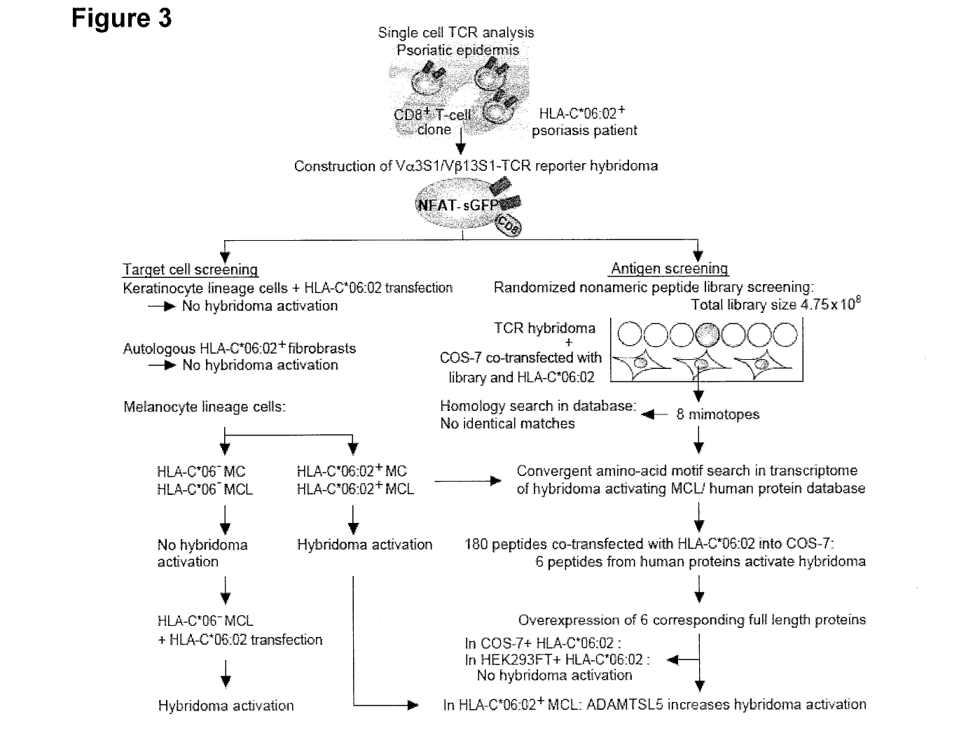

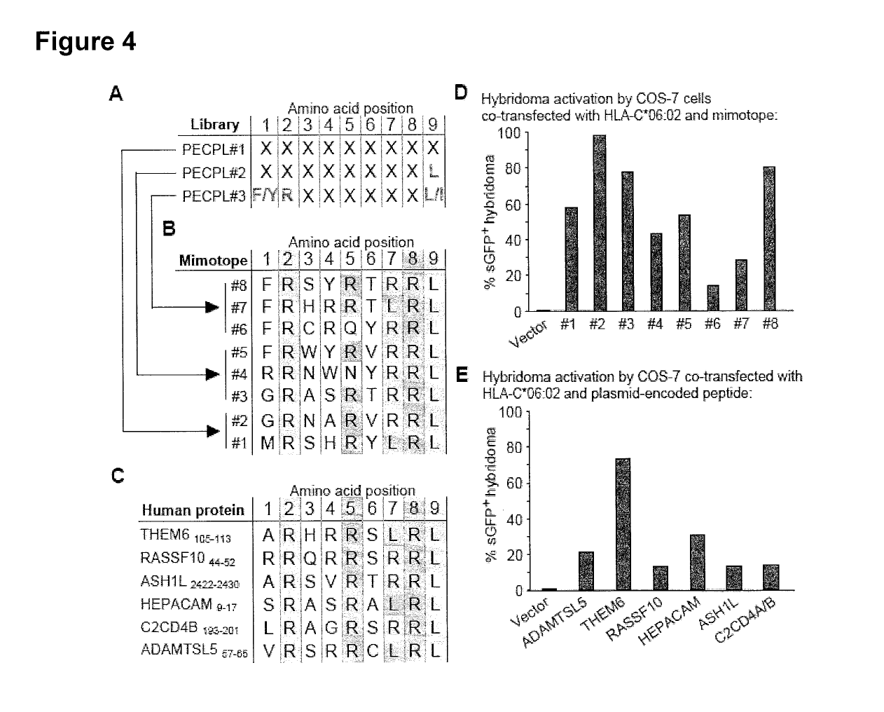

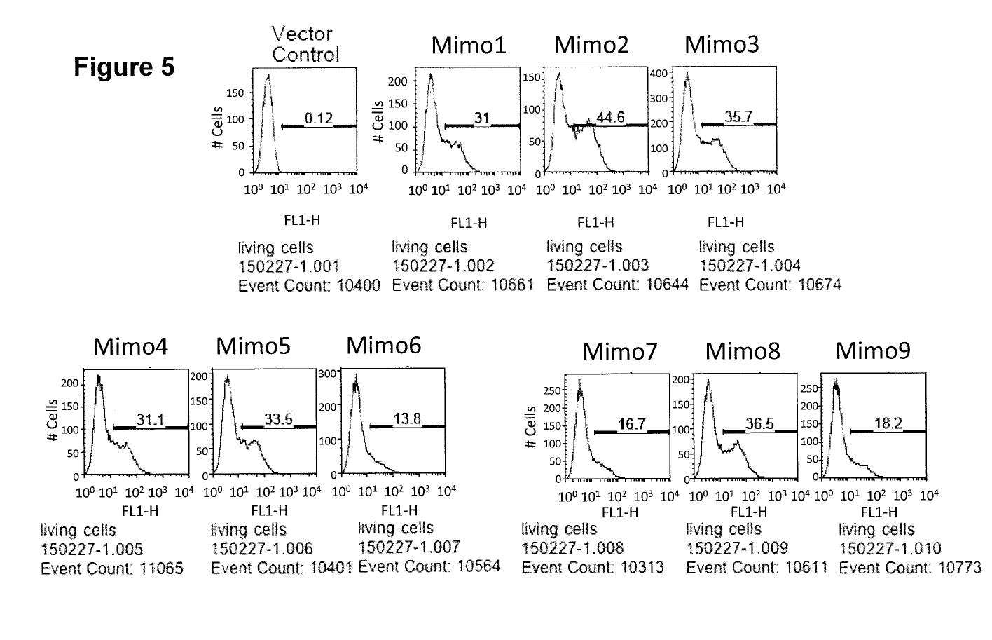

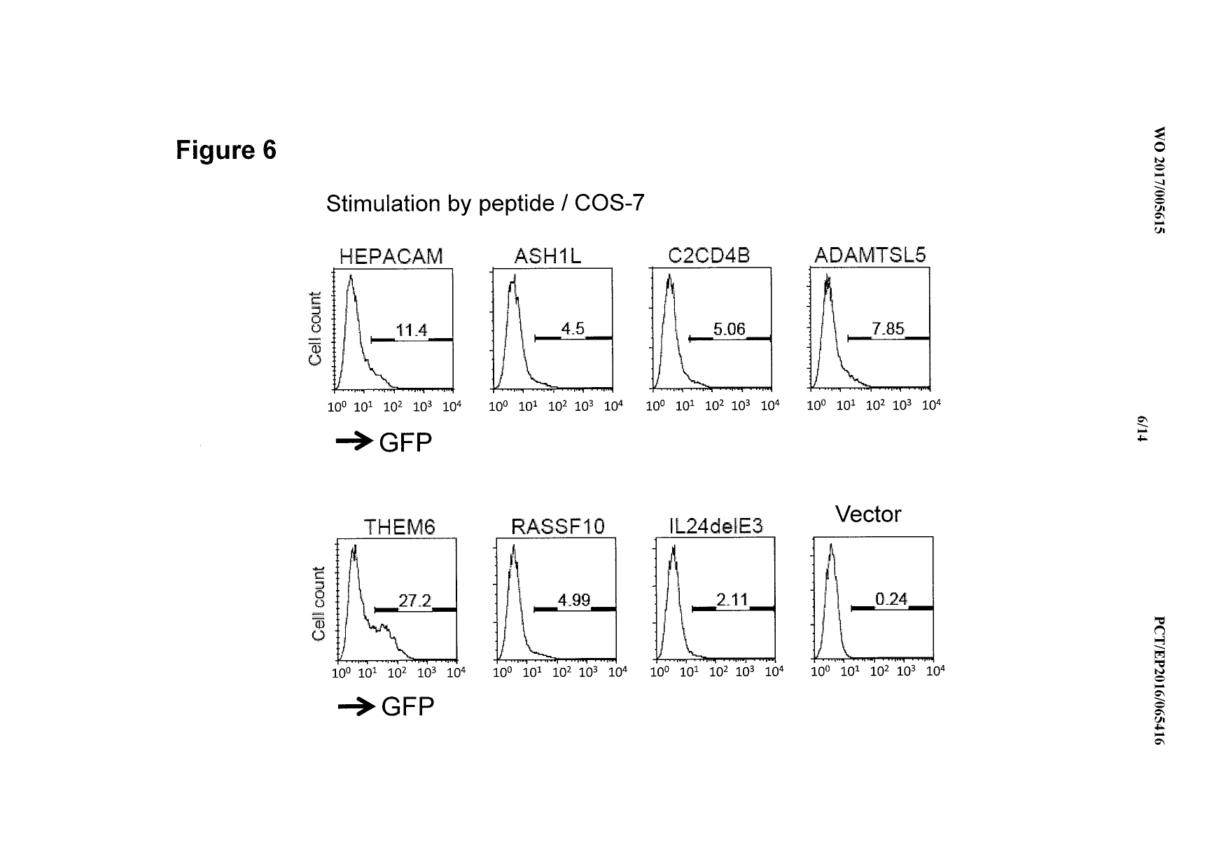

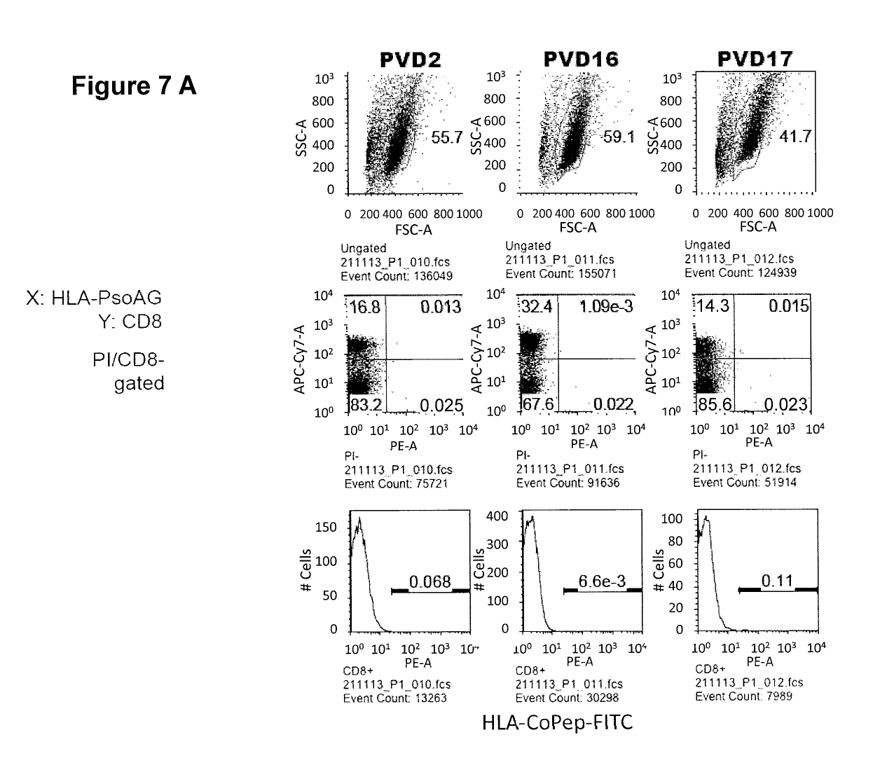

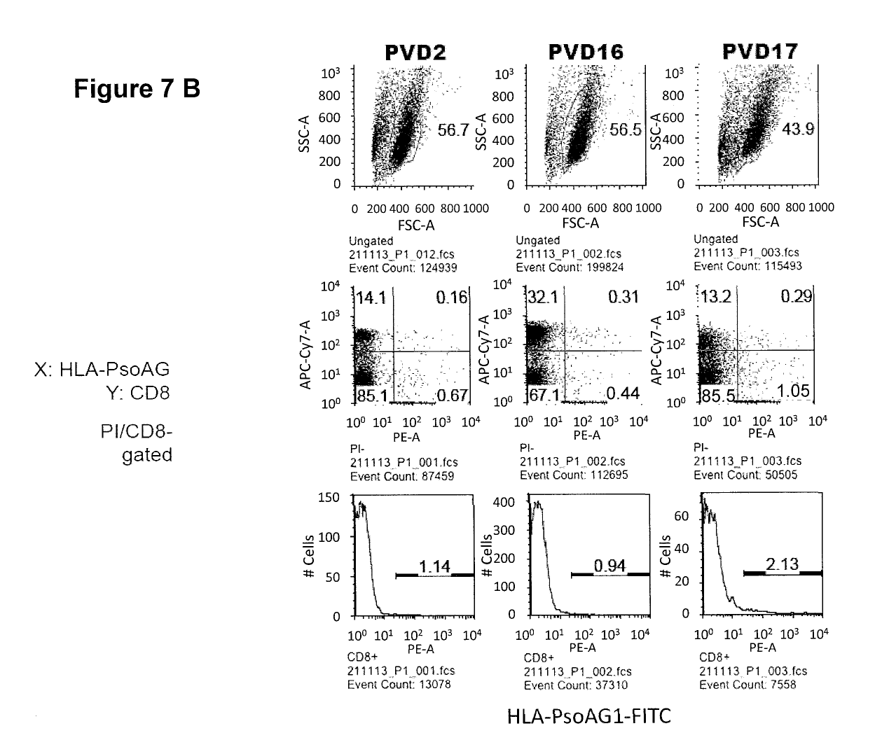

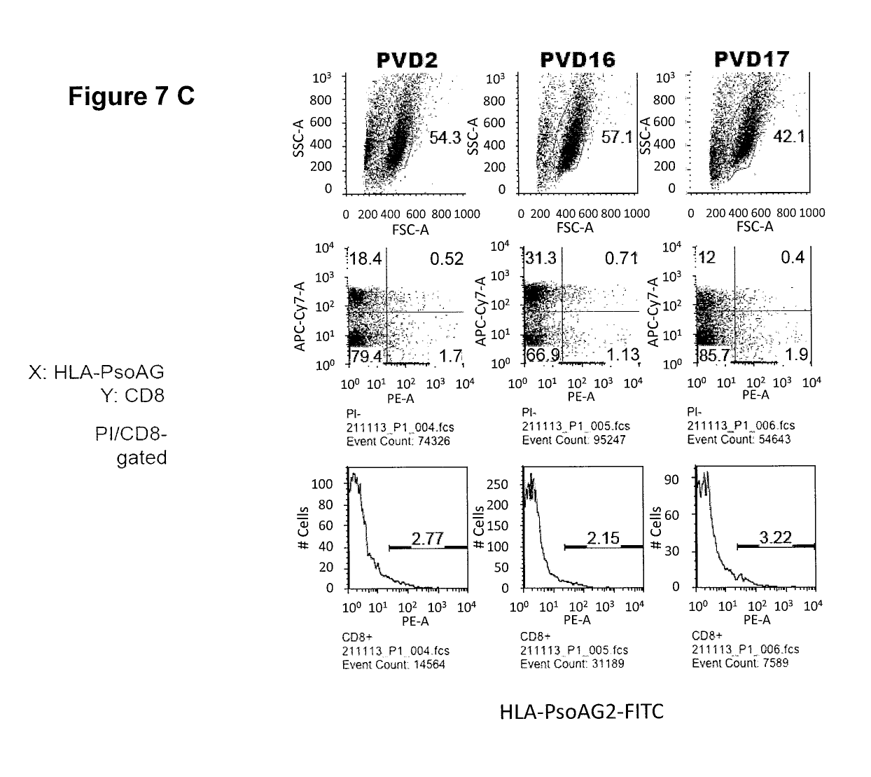

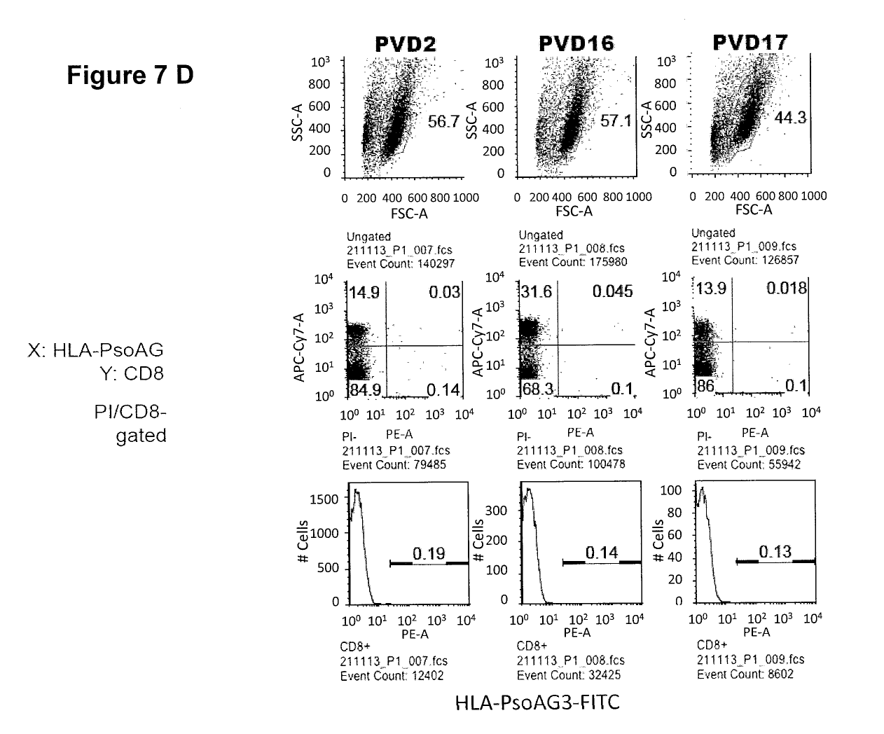

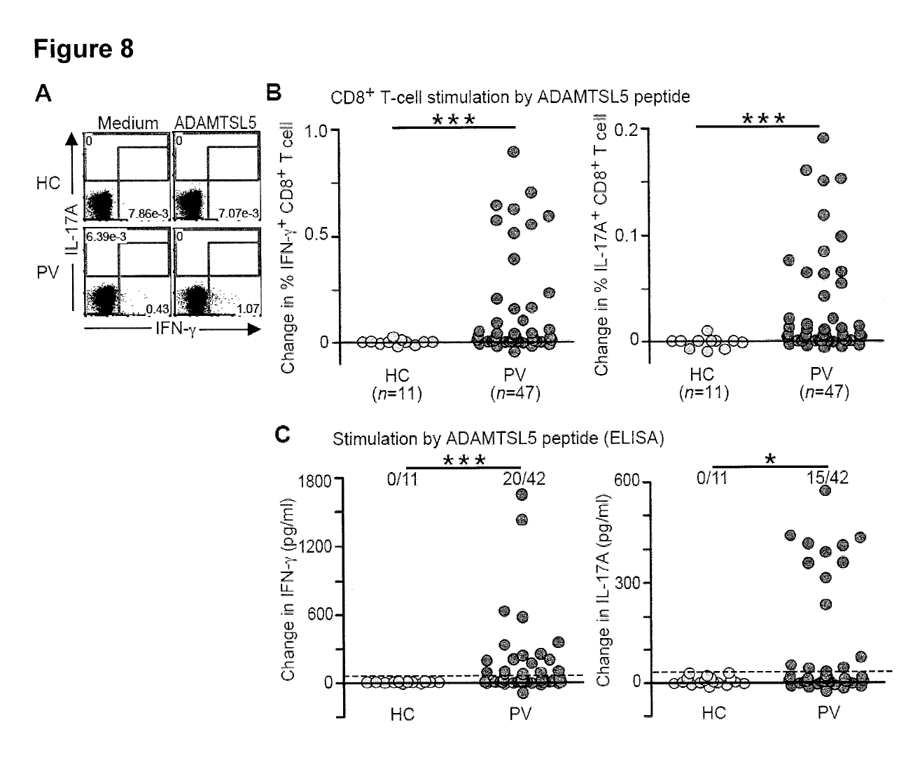

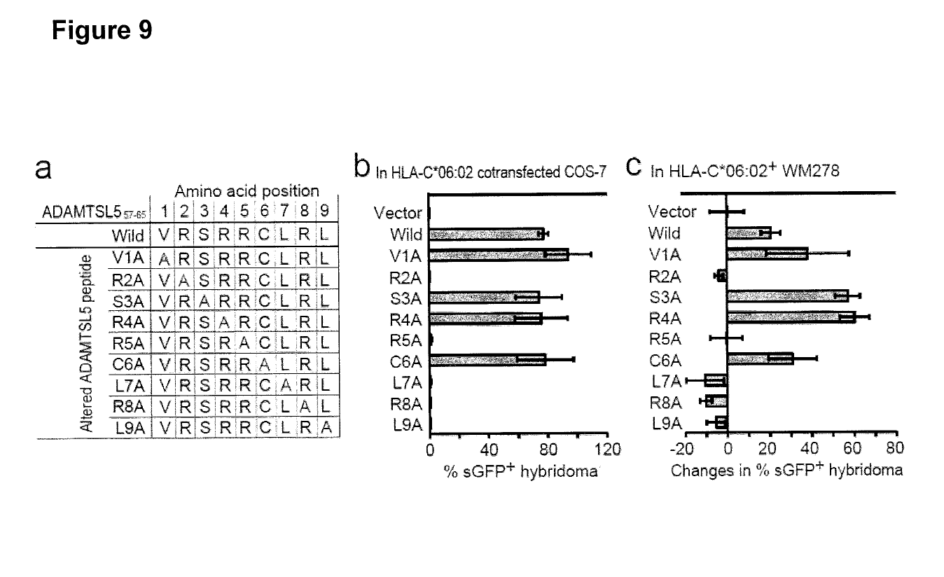

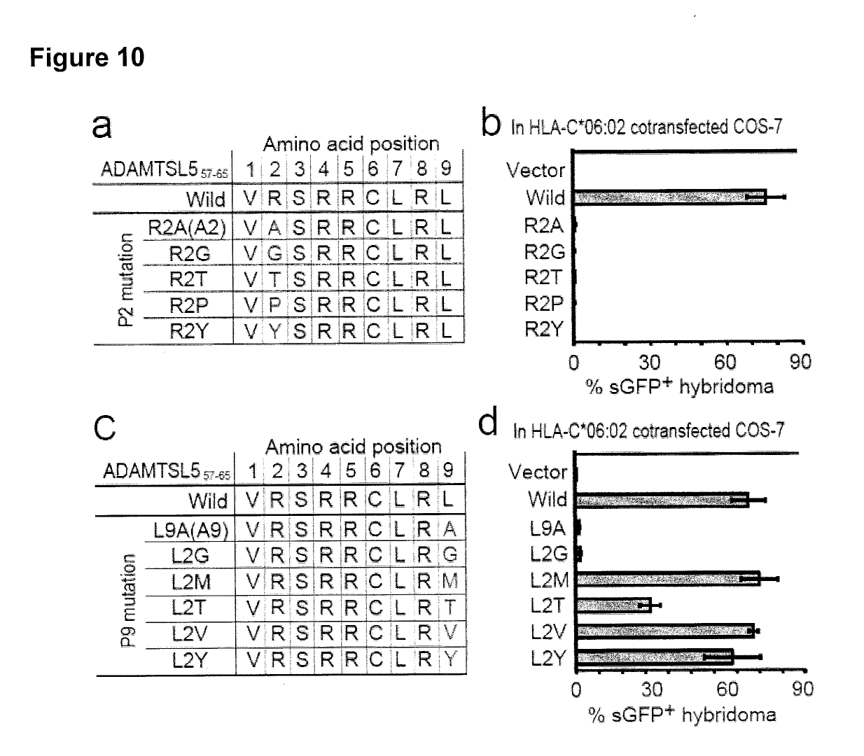

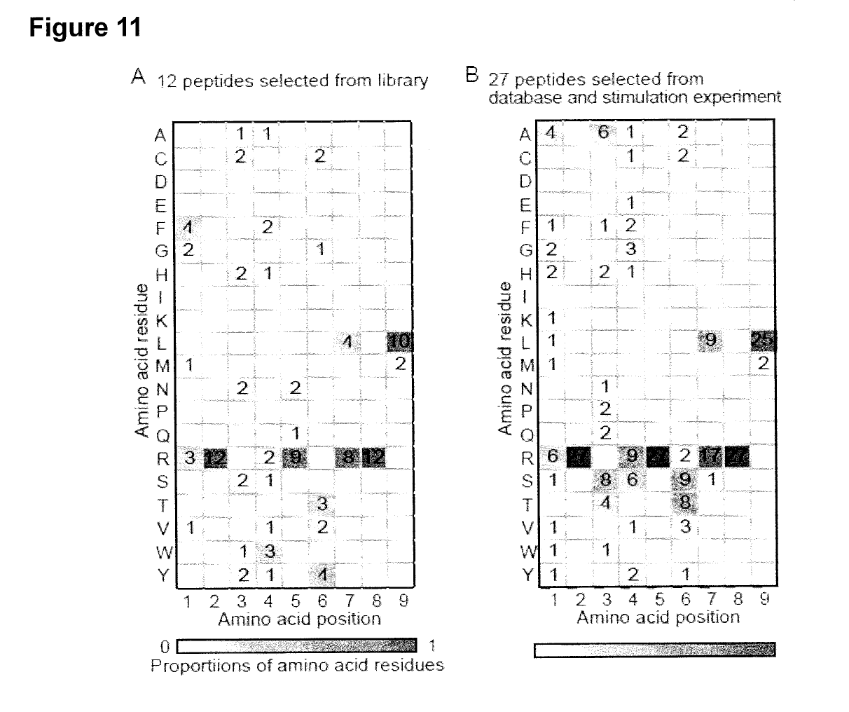

(A) TCR-hybridoma activation by primary human melanocytes (MC, elongated cells). Merged light and fluorescence microscopy images of Vα3S1/Vβ13S1-TCR hybridoma cells (round shapes) that were activated to express sGFP upon co-culture with HLA-C*06:02-positive (left) but not HLA-C*06:02-negative melanocytes (right).

(B) TCR-hybridoma activation by HLA-C*06:02-positive (left) or HLA-C*06:02-negative (right) primary human melanocytes (MC) in the absence or presence of IFN-γ or a HLA-class I blocking antibody. Frequency of NFAT-sGFP⁺ hybridoma cells was assessed by flow cytometry and normalized to the maximum value induced by CD3 activation in the same experiment. Data are representative of three HLA-C*06:02-positive (healthy n=2, psoriasis n=1) and nine HLA-C*06:02-negative individuals (healthy n=7, psoriasis n=2).

(C) Western blot analysis of HLA-C (upper panel) and β-actin expression (loading control, lower panel).in lysates from primary human melanocytes (MC, left) or the melanoma-derived cell line WM278 (right) cultured in the absence or presence of IFN-γ.

(D) TCR-hybridoma activation by co-culture with the HLA-C*06:02-positive melanocytic cell line (MCL) WM278 preincubated without or with IFN-γ or a HLA-class I blocking antibody. Data were assessed as in (B).

(E) TCR-hybridoma activation by HLA-C*06:02-negative melanocytic cell line (MCL) WM9 transfected with HLA-C*06:02 or HLA-A*0201 and incubated with an anti-HLA-class I antibody. Data were assessed as in (B).

(F) Western blot analysis of HLA-C (upper panel) and β-actin expression (loading control, lower panel) in lysates of WM9 cells transfected with pRSV-HLA-C*06:02.

Data represent 3 (C), >3 (D), and 3 (E) independent experiments.

FIG. 3. Screening Strategy to Determine the Vα3S1/Vβ13S1 TCR Reactivity. Schematic of screening strategy to identify cellular targets (left) or antigens (right) of the Vα3S1/Vβ13S1-TCR. Target cell screening involved co-cultured with different skin cell types. MC, primary human melanocytes. Antigen screening by nonamer peptide libraries was followed by transfection of candidate peptide epitopes and full-length proteins into HLA-C*06:02-transfected HEK293FT or COS-7 cells, or HLA-C*06:02-positive melanoma cell lines (MCL).

FIG. 4. Identification of Peptide Ligands of the Vα3S1/Vβ13S1 TCR.

(A) Design of plasmid-encoded combinatorial nonamer peptide libraries (PECPL) #1-3 with predefined amino acid residues in single letter amino acid code. X: randomized residue.

(B) Mimotopes derived from PECPL#1-3 activating the Vα3S1/Vβ13S1-TCR hybridoma. HLA-C*06:02 anchor positions are labelled in yellow, conserved amino acids of mimotopes independent from anchor positions in green, blue or red. See also FIG. S3.

(C) TCR-hybridoma activation by mimotopes co-transfected with HLA-C*06:02 into COS-7 cells. Frequency of NFAT-sGFP^{±}hybridoma cells was assessed by flow cytometry and normalized to the maximum value induced by CD3 activation in the same experiment.

(D) Natural human peptide ligands activating the Vα3S1/Vβ13S1-TCR hybridoma. Subscript numbers give amino acid positions in parent proteins. Labelling as in (B).

(E) TCR-hybridoma activation by plasmid-encoded natural human peptide epitopes co-transfected with HLA-C*06:02 into COS-7 cells. Data were assessed as in (C).

FIG. 5. Stimulation of Vα3S1/Vβ13S1 TCR hybridoma cells by mimotopes 1-9 co-transfected with HLA-C*06: 02 into COS-7 cells.

Flow cytometry analysis of NFAT-sGFP⁺ Vα3S1/Vβ13S1 TCR hybridoma cells following co-culture with COS-7 cells co-transfected with plasmids containing mimotopes and HLA-C*06:02.

FIG. 6. Stimulation of Vα3S1/Vβ13S1 TCR hybridoma cells by natural human peptide ligands co-transfected with HLA-C*06:02 into COS-7 cells.

Flow cytometry analysis of NFAT-sGFP⁺ Vα3S1/Vβ13S1 TCR hybridoma cells following co-culture with COS-7 cells co-transfected with plasmids containing natural human peptide epitopes and HLA-C*06:02.

FIG. 7. Staining of peripheral blood CD8⁺ T cells with peptide-HLA-C*06:02 dextramers.

(A) HLA-C*06:02/control peptide

(B) HLA-C*06:02/RASSF10 peptide

(C) HLA-C*06:02/HEPACAM peptide

(D) HLA-C*06:02/IL24del3 peptide

Shown is the FACS analysis of CD8⁺ T cells stained with labelled peptide/HLA-C*06:02-dextramers. HLA-C*06:02/control peptide refers to an unrelated peptide formerly isolated from HLA-C*06:02.

FIG. 8. Cytokine Induction by Stimulation of PBMC with ADAMTSL5 Peptide.

(A) Intracellular staining of IFN-γ and IL-17A in CD8⁺ T cells following stimulation of PBMC with synthetic ADAMTSL5 peptide (right) or medium control (left). Representative histograms show flow cytometry analysis of each a representative experiment for a healthy control (HC) and a psoriasis patient (PV).

(B) Changes in frequencies of IFN-γ⁺- and IL-17A⁺CD8⁺ T cells after ADAMTSL5 peptide stimulation of PBMC from healthy controls (HC, n=11) and psoriasis patients (PV, n=47) after normalization to medium control. Each dot represents one individual. ***p<0.005 (Mann-Whitney U test).

(C) Changes in IFN-γ (left) and IL-17A concentrations (right) in culture supernatants of PBMC from healthy controls (HC, n=11) or psoriasis patients (PV, n=37) following stimulation with synthetic ADAMTSL5 peptide as determined in triplicates by ELISA. Graphs depict changes by ADAMTSL5 peptide over medium control, each dot represents one individual. Dashed line indicates threshold. ***p<0.005, **p<0.01 (Chi square test).

FIG. 9. Alanine scan of peptide positions in the ADAMTSL5 peptide.

Exchange of amino acids at positions 1 to 9 (a) of the ADAMTSL5 peptide against Ala. Activation of Vα3S1/Vβ13S1 TCR hybridoma by mutated peptides expressed together with HLA-C*06:02 in COS-7 (b) or WM278 cells (c) is determined by FACS analysis of green fluorescent hybridoma cells.

FIG. 10. Validation of amino acids at P2 (R) and P9 (Z) in the ADAMTSL5 nonameric peptide.

Exchange of amino acids at position 2 (a) and position (c) of the ADAMTSL5 peptide. Activation of Vα3S1/Vβ13S1 TCR hybridoma by mutated peptides co-transfected with HLA-C*06:02 in COS-7 cells is deteimined by FACS analysis of green fluorescent hybridoma cells (b and d).

FIG. 11. Definition of amino acids at position 1 to 9 of nonamer peptides stimulating the Vα3S1/Vβ13S1 T-cell receptor.

(A) Amino acids at position 1 to position 9 of 12 nonamer mimotopes identified by library screening.

(B) Amino acids at position 1 to position 9 of 27 octamer or nonamer peptides identified by database screening. Numbers give the frequency of amino acids identified at a particular position.

### EXAMPLES

1. Experimental Procedures

1.1 Human Subjects

Lesional biopsies were obtained from patients with chronic plaque psoriasis. Blood samples were obtained by antecubital venous puncture. All participants gave written infoiiiied consent. The study was performed in accordance with the Helsinki Declaration and approved by the Ethics committee of the university hospital 1.2 Generation of Vα3S1/Vβ13S1-TCR CD8⁺ reporter T-hybridoma

Identification of the matching Vα3S1/Vβ13S1-TCR chains of a CD 8⁺ T-cell clone (Vα3S1-NN-Jα 45.1: CA TDAL YSGG, VP13S1-N(D)N-Jβ 3 1.1: CASSY SEGED EAFF; Arden nomenclature, see Arden et al. 1995) has been described (see Kim et al. 2012). TCR-hybridoma generation was perfoinied as described (Seitz et al., 2006; Siewert et al., 2011). Briefly, Vα and Vβ-regions were cloned into expression plasmids pRSV-hygro (α-chain) and pRSV-neo (β-chain) using restriction sites SalI-PvuII or SalI-BIpI. The resulting plasmids were linearized (XmnI and NdeI, respectively) and electroporated into 58 α⁻β⁻ T hybridoma cells. Functional clones were supertransfected with pLPCX-CD8α-IRES-β and pcDNA-NFAT-sGFP. TCR-activation induced NFAT-sGFP-expression was determined by CD3 crosslinking with anti-mouse CD3 antibody (clone 17A2, eBioscience), flow cytometry and fluorescence microscopy (AxioVert200M, Zeiss, 520/35 BrightLine filter, Semrock and 605/70 filter). Clones with highest frequencies of responding cells (usually >30% NFAT-sGFP-positive cells after CD3 stimulation) were expanded in T-hybridoma medium (see “Primary cells and cell lines” section). Hybridoma batches were frequently recloned to minimize sporadic sGFP expression and decrease of activation rates. The resulting cell line is teimed Vα3S1/Vβ13S1-TCR hybridoma.

1.3 Construction of Plasmid-Encoded Combinatorial Peptide Libraries (PECPL) and Identification of Vα3S1/Vβ13S1-TCR Mimotopes

Completely randomized nonameric PECP library #1, PECPL #2 with predetermined amino acids Leu at P9 or #3 with Phe/Tyr at P1, Arg at P2 and Leu/Ile at P9 were prepared as described⁴. Screening strategy and library designs and are shown in FIG. 3 and FIG. 4. COS-7 cells were co-transfected with PECP-libraries and HLA-C*06:02, co-cultured with Vα3S1/Vβ13S1-TCR hybridoma and screened by UV-microscopy for the induction of sGFP. Mimotope-containing plasmids were isolated and sequenced.

PECPL screening was performed as described (Siewert et al., 2011). COS-7 cells were cotransfected with PECPLs and HLA-C*06:02 and cocultured with Vα3S1/Vβ13S1-TCR hybridoma cells. After 16 h, COS-7 cells in close contact to fluorescent hybridoma cells were isolated and library peptides were amplified by nested PCR. PCR products were cloned into pcDNA.3.1DN5-His-TOPO and transformed into E. coli. The mimotope-enriched library plasmids were cotransfected with pRSV-HLA-C*06:02 into COS-7 cells and the mimotope-containing plasmid was isolated by subcloning and sequencing.

Amino acid sequences of mimotopes were aligned and used in different search strategies to identify corresponding peptides from human proteins in the taxa-specific homo sapiens [9606] UniProt database or melanocytic transcriptome. cDNA corresponding to 180 candidate peptide antigens or six corresponding full-length proteins were cloned into pcDNA3.1DN5-His-TOPO.

For this purpose, forward and reverse oligonucleotides coding for octamer or nonamer candidate peptides were annealed in 1× Pwo buffer at a concentration of 10 pmol/μl by heating to 95° C. for 5 min and then slowly cooled to room temperature. Diluted annealing products were ligated into pcDNA3.1D/V5-His-TOPO using the Directional TOPO Expression Kit. Forward primers carried a 5′-CACCATG overhang and a stop codon at 3′-end of the target sequence. Resulting constructs were sequence verified. Oligonucleotides for cloning of ADAMTSL5 are given as example for the design.

ADAMTSL5-peptide for:
[SEQ ID NO. 77]
5′-caccatggtgcggagccggcggtgcctgcggctgtga-3′
ADAMTSL5-peptide rev:
[SEQ ID NO. 78]
5′-tcacagccgcaggcaccgccggctccgcac-3′

For Ala scans we generated plasmids encoding the sequence of the ADAMTSL5 peptide with exchanges of each one amino acid of the natural peptide sequence at position P1 to P9 against nucleotide triplets encoding the amino acid Ala. To verify amino acids at P2 and P9, the amino acids at P2 and P9 were exchanged against nucleotide triplets encoding the amino acids Ala, Gly, Thr, Pro or Tyr (P2) or against Ala, Gly, Met, Thr, Val or Tyr (P9) accordingly.

Ectopic protein expression was verified by Western blotting. Mutations of ADAMTSL5 epitope₅₈₋₆₅ were introduced by PCR using internal reverse primers. siRNAs were transfected into WM278 cells for ADAMTSL5 knock down. Knock down efficacy was examined by triplicate quantitative PCR using PBGD as an internal standard.

1.4 Cells, Cell Lines and Vα3S1/Vβ13S1-TCR Hybridoma Activation Assays

Primary human melanocytes were cultured from skin samples (Hsu and Herlyn, 1996). Peripheral blood mononuclear cells were prepared by density gradient centrifugation. Human neonatal epidermal keratinocytes were obtained commercially. The following cell lines were used: COS-7, HEK293FT, HaCaT, A431, WM278, WM9, WM239A, 1205Lu.

Activation of Vα3S1/Vβ13S1-TCR hybridoma cells was assessed in co-culture experiments by NFAT-sGFP induction and determined by flow cytometry and UV-fluorescence microscopy. IFN-γ was added to increase HLA-class I expression of HLA-C*06:02-positive cells/cell lines. HLA-C*06:02-negative cells or cell lines were transfected with pRSV-HLA-C*06:02 or pRSV-HLA-A*0201. Cell lines were transfected with plasmid-encoded peptides or full-length proteins. HLA-class I antibody was used to block antigen-mediated TCR ligation.

sGFP induction in Vα3S1/Vβ13S1-TCR hybridoma cells was examined after 24 h coculture with antigen-presenting cells by UV-fluorescence microscopy and/or flow cytometry. As controls, Vα3S1/Vβ13S1-TCR hybridoma cells were cultured in anti-mouse CD3 antibody-coated (clone 17A2, 2 μg/ml in PBS, eBioscience) culture plates. pRSV-GFP or pcDNA-GFP served as transfection controls.

HLA-C*06:02-negative antigen-presenting cells or cell lines were transfected with pRSV-HLA-C*06:02 or pRSV-HLA-A*0201. Cell lines were transfected with plasmid-encoded peptides or full-length proteins. HLA-class I antibody was used to block antigen-mediated TCR ligation.

1.5 Identification of Natural Candidate Peptide Antigens Based on Mimotope Sequences

Two different search strategies were used to identify human candidate peptide epitopes based on the conserved amino acid motifs of the mimotopes. We employed two tools from the “The European Molecular Biology Open Software Suite” (http://emboss.sourceforge.net/): “PROPHECY” (http://emboss.sourceforge.net/apps/release/6.6/emboss/apps/prophecy.html) was used to create a frequency matrix from sequences of positively tested antigenic peptides. With this matrix and the “PROFIT” tool (http://emboss.sourceforge.net/apps/release/6.6/emboss/apps/profit.html), the taxa-specific Homo sapiens [9606] UniProt database was scanned. The list of results was further refined with infoiniation on anchor amino acids of HLA-C*06:02 ligands (see Dionne et al., 2004; Falk et al., 1993) (Syfpeithi database at http://www.syfpeithi.de/). The matrix was constantly adjusted according to newly isolated mimotopes or tested candidate antigen peptides

1205Lu transcriptome data were used for a selective search in melanocytic proteins. The MOTIF Search web server (http://www.genome.jp/tools/motif/MOTIF2.html) was searched for human genes in the “KEGG Genes” dataset containing peptide motifs that were defined according to mimotope sequences and general HLA-C*06:02 anchor positions.

To identify peptides from pathogens or food components which could activate the Vα3S1/Vβ13S1 TCR we searched the sequences of the mimotopes and the sequences of the human peptides ligating the Vα3S1/Vβ13S1 TCR against all known protein sequences or sequence specified according to bacterium sp. (taxid:77133), viruses (taxid:10239), fungi (taxid:4751) using the Protein BLAST search tool at: blast.ncbi nlm nih.gov/Blast.cgi?PAGE=Protein. Candidate peptides were cloned into pcDNA3.1DN5-His-TOPO as described above and tested in cotransfection experiments with HLA-C*06:02 for their ability to activate the Vα3S1/Vβ13S1 TCR hybridoma. Hybridoma activation was verified by measuring induction of green fluorescence protein in flow cytometry.

1.6 Dextramer Staining

Fluorescent dextramers were prepared of HLA-C*06:02 and antigenic or control peptides. The control peptide has been originally described as an HLA-C*06:02-ligand by Falk et al. (1993). PBMC from psoriasis patients were incubated with standard dilutions of the HLA-C*06:02-peptide dextramers and a phycoerythrin-labelled CD8 antibody and the frequency of double stained cells subsequently determined by flow cytometry on a FACScan flow cytometer. Data were analyzed by FlowJo software 887.

1.7 Peptide Stimulation of PBMC

PBMC were stimulated with synthetic peptides (10 ng/ml) for 48 hrs. Intracellular cytokine staining (Fujii et al., 2011) employed antibodies against CD8, IL-17A and IFN-γ. Data were acquired by flow cytometry and analysed by Flow Jo software. INF-γ and IL-17A levels in culture supernatants were determined by ELISA.

1.8 Statistics

Kruskal-Wallis H-test was used for multiple comparisons and Bonferroni correction was applied. Whenp-value of Kruskal-Wallis H-test was significant, unpaired data of two groups were compared using Mann-Whitney U-test. Differences in the two groups were compared with Fisher's exact test. Two-tailed p-values<0.05 were considered significant.

The features disclosed in the foregoing description, in the claims and/or in the accompanying drawings may, both separately and in any combination thereof, be material for realizing the invention in diverse forms thereof.

### REFERENCES

Altman J D, Moss P A, Goulder P J, et al. Phenotypic analysis of antigen-specific T lymphocytes. Science 1996; 274(5284): 94-96.

Arden, B., Clark, S. P., Kabelitz, D., and Mak, T. W. 1995. Human T-cell receptor variable gene segment families. Immunogenetics 42:455-500.

Birnbaum, M. E., Mendoza, J. L., Sethi, D. K., Dong, S., Glanville, J., Dobbins, J., Ozkan, E., Davis, M. M., Wucherpfennig, K. W., and Garcia, K. C. 2014. Deconstructing the peptide-MHC specificity of T cell recognition. Cell 157:1073-1087.

Boukamp, P., Petrussevska, R. T., Breitkreutz, D., Hornung, J., Markham, A., and Fusenig, N. E. 1988. Normal keratinization in a spontaneously immortalized aneuploid human keratinocyte cell line. J Cell Biol 106:761-771.

Chang, J. C., Smith, L. R., Froning, K. J., Kurland, H. H., Schwabe, B. J., Blumeyer, K. K., Karasek, M. A., Wilkinson, D. I., Farber, E. M., Carlo, D. J., et al. 1997. Persistence of T-cell clones in psoriatic lesions. Arch Dermatol 133:703-708.

Chiricozzi, A., and Krueger, J. G. 2013. IL-17 targeted therapies for psoriasis. Expert Opin Investig Drugs 22:993-1005.

Cole D K, Miles K M, Madura F, Holland C J, Schauenburg A J, Godkin A J, Bulek A M, Fuller A, Akpovwa H J, Pymm P G, Liddy N, Sami M, Li Y, Rizkallah P J, Jakobsen B K, Sewell AK.T-cell receptor (TCR)-peptide specificity overrides affinity-enhancing TCR-major histocompatibility complex interactions. J Biol Chem. 2014;289(2):628-38.

Conrad, C., Boyman, O., Tonel, G., Tun-Kyi, A., Laggner, U., de Fougerolles, A., Kotelianski, V., Gardner, H., and Nestle, F.O. 2007. Alphalbetal integrin is crucial for accumulation of epidermal T cells and the development of psoriasis. Nat Med 13:836-842.

Dal Porto J, Johansen T E, Catipovio B, et al. A soluble divalent class I major histocompatibility complex molecule inhibits alloreactive T cells at nanomolar concentrations. Proc Natl Acad Sci USA 1993; 90(14): 6671-6675.

Davidson, A., and Diamond, B. 2001. Autoimmune diseases. N Engl J Med 345:340-350.

Davis, M. M., and Bjorkman, P. J. 1988. T-cell antigen receptor genes and T-cell recognition. Nature 334:395-402.

Di Cesare, A., Di Meglio, P., and Nestle, F. O. 2009. The IL-23/Th17 axis in the immunopathogenesis of psoriasis. J Invest Dermatol 129:1339-1350.

Dionne, S. O., Lake, D. F., Grimes, W. J. & Smith, M .H 2004. Identification of HLA-Cw6.02 and HLA-Cw7.01 allele-specific binding motifs by screening synthetic peptide libraries. Immunogenetics 56, 391-398

Falk, K., Rotzschke, O., Grahovac, B., Schendel, D., Stevanovic, S., Gnau, V., Jung, G., Strominger, J. L., and Rammensee, H.G. 1993. Allele-specific peptide ligand motifs of HLA-C molecules. Proc Natl Acad Sci USA 90:12005-12009.

Fujii, H., Arakawa, A., Kitoh, A., Miyara, M., Kato, M., Kore-eda, S., Sakaguchi, S., Miyachi, Y., Tanioka, M., and Ono, M. (2011). Perturbations of both nonregulatory and regulatory FOXP3+ T cells in patients with malignant melanoma. Br J Dermatol 164, 1052-1060.

Griffiths, C. E., and Barker, J. N. 2007. Pathogenesis and clinical features of psoriasis. Lancet 370:263-271.

Hsu, M. Y., and Herlyn, M. (1996). Cultivation of normal human epidetmal melanocytes. Methods in molecular medicine 2, 9-20.

Icen, M., Crowson, C. S., McEvoy, M. T., Dann, F. J., Gabriel, S. E., and Maradit Kremers, H. 2009. Trends in incidence of adult-onset psoriasis over three decades: a population-based study. J Am Acad Dermatol 60:394-401.

Kent, S. C., Chen, Y., Bregoli, L., Clemmings, S. M., Kenyon, N. S., Ricordi, C., Hering, B. J., and Hafler, D. A. 2005. Expanded T cells from pancreatic lymph nodes of type 1 diabetic subjects recognize an insulin epitope. Nature 435:224-228.

Kim, S. M., Bhonsle, L., Besgen, P., Nickel, J., Backes, A., Held, K., Vollmer, S., Dornmair, K., and Prinz, J. C. 2012. Analysis of the Paired TCR alpha- and beta-chains of Single Human T Cells. PLoS ONE 7:e37338.

Nair, R. P., Stuart, P. E., Nistor, I., Hiremagalore, R., Chia, N. V., Jenisch, S., Weichenthal, M., Abecasis, G. R., Lim, H. W., Christophers, E., et al. 2006. Sequence and haplotype analysis supports HLA-C as the psoriasis susceptibility 1 gene. Am J Hum Genet 78:827-851.

Neefjes, J., Jongsma, M. L., Paul, P., and Bakke, O. 2011. Towards a systems understanding of MHC class I and MHC class II antigen presentation. Nat Rev Immunol 11:823-836.

Parisi, R., Symmons, D. P., Griffiths, C. E., and Ashcroft, D. M. 2013. Global epidemiology of psoriasis: a systematic review of incidence and prevalence. J Invest Dermatol 133:377-385.

Scholler, J. et al. A recombinant human HLA-class I antigen linked to dextran elicits innate and adaptive immune responses. J. Immunol. Methods 360, 1-9 (2010).

Seitz, S., Schneider, C. K., Malotka, J., Nong, X., Engel, A. G., Wekerle, H., Hohlfeld, R., and Dornmair, K. 2006. Reconstitution of paired T cell receptor alpha- and beta-chains from microdissected single cells of human inflammatory tissues. Proc Nati Acad Sci U S A 103:12057-12062.

Siewert, K., Malotka, J., Kawakami, N., Wekerle, H., Hohlfeld, R., and Dornmair, K. 2011. Unbiased identification of target antigens of CD8+T cells with combinatorial libraries coding for short peptides. Nat. Medicine 18:824-828.

Tan M P, Gerry A B, Brewer J E, Melchiori L, Bridgeman J S, Bennett A D, Pumphrey N J, Jakobsen B K, Price D A, Ladell K, Sewell A K. T cell receptor binding affinity governs the functional profile of cancer-specific CD8+ T cells. Clin Exp Immunol. 2015; 180(2):255-70.

Uchanska-Ziegler, B., Loll, B., Fabian, H., Hee, C. S., Saenger, W., and Ziegler, A. 2012. HLA class I-associated diseases with a suspected autoimmune etiology: HLA-B27 subtypes as a model system. Eur J Cell Biol 91:274-286.

Wucherpfennig, K. W., Allen, P. M., Celada, F., Cohen, I. R., De Boer, R., Garcia, K. C., Goldstein, B., Greenspan, R., Hafler, D., Hodgkin, P., et al. 2007. Polyspecificity of T cell and B cell receptor recognition. Semin Immunol 19:216-224.

1. A peptide comprising an amino acid sequence of the general formula I
X1ₙ-Arg-X2-X3-Y1-X4-Y2-Arg-Z (I)
wherein
X1 is selected from Phe, Arg, Gly, Met, Ala, Ser, Leu and Val;
n is 0 or 1.
X2 is any amino acid;
X3 is any amino acid;
X4 is selected from Thr, Tyr, Val, Cys, Ser, and Ala;
Y1 is selected from Arg, Lys, Gln, and Asn;
Y2 is selected from Arg, Leu or and Ser; and
Z is selected from Leu, Met, Ile or and
or
wherein
X1 is selected from Phe, Arg, Gly, Met, Ala, Ser, Leu, Val, His, Tyr, Lys and Trp;
n is 0 or 1.
X2 is any amino acid;
X3 is any amino acid;
X4 is selected from Thr, Tyr, Val, Cys, Ser, Ala, Gly and Arg;
Y1 is selected from Arg, Lys, Gln, and Asn;
Y2 is selected from Arg, Leu and Ser; and
Z is selected from Leu, Met, Ile, Val, Thr and Tyr.

2. The peptide of claim 1, wherein the peptide has a length of 8 to 150 amino acids.

3. The peptide of claim 1, further comprising one or more further components selected from:
N- and/or C-terminal modifications;
labels;
tags;
drugs or their respective prodrugs;
carriers or depots for drugs, prodrugs or labels;
immunogenic epitopes;
cytotoxins;
radionuclides,
and/or combinations thereof.

4. The peptide of claim 1, wherein
X2 is selected from Ser, His, Cys, Trp, Asn, Ala, Tyr, Gln, Phe, Thr, and Pro;
and/or
X3 is selected from Tyr, Arg, Trp, Ser, Ala, His, Phe, Val, Gly, Cys and Glu;
and/or
X1, if present, is selected from Phe, Arg, Gly, Met, Ala, Ser, Leu, Val, His, Tyr, Lys and Trp;
and/or
X4 is selected from Thr, Tyr, Val, Cys, Ser, Ala, Gly and Arg
and/or
Y2 is selected from Arg, Leu and Ser;
and/or
Z is selected from Leu, Met, Ile, Val, Thr and Tyr.

5. The peptide of claim 1, comprising an amino acid sequence selected from the following formulas
X1-Arg-X2-X3-Y1-X4-Y2-Arg-Leu (Ia)
X1-Arg-X2-X3-Y1-X4-Arg-Arg-Z (Ib) and
X1-Arg-X2-X3-Y1-X4-Arg-Arg-Leu (Ia)
wherein
X1, X2, X3, X4 and Y1 are as defined in claim 1;
Y2 is selected from Arg, and Leu; and
Z is selected from Leu, Met, Ile and Val;
and/or wherein the peptide comprises or consists of an amino acid sequence selected from the group of SEQ ID NOs. 1 to 13, 17 to 19 and 32 to 57.

6. A peptide-HLA-C complex selected from:
a) a peptide-HLA-C monomer complex,
comprising
(i) at least one peptide selected from a peptide of claim 1 or a peptide comprising or consisting of an amino acid sequence of SEQ ID NOs: 14-16, and
(ii) a HLA-C monomer that binds to a T-cell receptor (TCR); and
b) a peptide-HLA-C multimer complex, comprising at least one peptide selected from a peptide of claim 1 or a peptide comprising or consisting of an amino acid sequence of SEQ ID NOs: 14-16, and at least two HLA-C monomers that bind to a TCR.

7. (canceled)

8. The complex of claim 6, wherein the HLA-C comprises one or more further components selected from:
labels;
tags;
drugs or their respective prodrugs;
carriers or depots for drugs, prodrugs or labels;
immunogenic epitopes;
cytotoxins;
radionuclides;
coupling moiety/moieties;
and combinations thereof.

9. The complex of claim 6, wherein the at least one peptide is altered for modifying the TCR binding affinity of the peptide or the peptide-HLA-C complex.

10. A pharmaceutical composition, comprising
(A) at least one peptide of claim 1 and/or a a peptide-HLA-C complex selected from:
a) a peptide-HLA-C monomer complex,
comprising
(i) at least one peptide selected from a peptide of claim 1 or a peptide comprising or consisting of an amino acid sequence of SEQ IDNOs: 14-16, and
(ii) a HLA-C monomer that binds to a T-cell receptor (TCR); and
b) a peptide-HLA-C multimer complex, comprising at least one peptide selected from a peptide of claim 1 or a peptide comprising or consisting of an amino acid sequence of SEQ ID NOs: 14-16, and at least two HLA-C monomers that bind to a TCR; and
a pharmaceutically acceptable carrier and/or excipient.

11. The pharmaceutical composition of claim 10, comprising two or more of said peptides.

12. A method for performing at least one of the following:
monitoring Psoriasis disease activity;
monitoring efficacy of Psoriasis treatment;
determining Psoriasis disease risk; and/or
determining the frequency of autoreactive T cells in samples of subjects having Psoriasis;
wherein said method comprises the use of:
a peptide of claim 1;
a peptide comprising or consisting of an amino acid sequence of SEQ ID NOs: 14-16;
a peptide-HLA-C complex selected from:
a) a peptide-HLA-C monomer complex,
comprising:
(i) at least one peptide selected from a peptide of claim 1 or a peptide comprising or consisting of an amino acid sequence of SEQ ID NOs: 14-16, and
(ii) a HLA-C monomer that binds to a T-cell receptor (TCR); and
b) a peptide-HLA-C multimer complex, comprising at least one peptide selected from a peptide of claim 1 or a peptide comprising or consisting of an amino acid sequence of SEQ ID NOs: 14-16, and at least two HLA-C monomers that bind to a TCR; or
a protein comprising an amino acid sequence of SEQ ID NOS: 22 to 31 or 60 to 76;

13. (canceled)

14. A method for the prevention and/or treatment of Psoriasis wherein said method comprises administering, to a subject in need of such prevention and/or treatment:
a peptide of claim 1;
a peptide comprising or consisting of an amino acid sequence of SEQ ID NOs:
14-16;
a peptide-HLA-C complex selected from:
a) a peptide-HLA-C monomer complex,
comprising
(i) at least one peptide selected from a peptide of claim 1 or a peptide comprising or consisting of an amino acid sequence of SEQ ID NOs: 14-16, and
(ii) a HLA-C monomer that binds to a T-cell receptor (TCR); and
b) a peptide-HLA-C multimer complex, comprising at least one peptide selected from a peptide of claim 1 or a peptide comprising or consisting of an amino acid sequence of SEQ ID NOs: 14-16, and at least two HLA-C monomers that bind to a TCR; or
a protein comprising an amino acid sequence of SEQ ID NOs: 22 to 31 or 60 to 76.

15-16. (canceled)

17. The method according to claim 14, wherein the prevention and/or treatment of Psoriasis, comprises
(a) inhibiting or blocking the interaction of TCR and HLA-C, and/or
(b) suppressing the expression of HLA-C.

18. The method according to claim 14, wherein the prevention and/or treatment of Psoriasis comprises:
creating immunotolerance against autoantigens;
affecting, tolerating or blocking the activation of autoreactive T cells for therapeutic purposes;
eliminating pathogenic CD8⁺ T cells; and/or
down-regulating a pathogenic immune response by specifically blocking HLA-C/T-cell-interactions.

19. (canceled)

20. A method for the prevention and/or treatment of Psoriasis, comprising
(a) inhibiting or blocking the interaction of TCR and HLA-C, and/or
(b) suppressing the expression of HLA-C.

21. The method according to claim 20, wherein
(a) inhibiting or blocking the interaction of TCR and HLA-C comprises interfering with the contact between TCR and HLA-C and/or
(b) suppressing the expression of HLA-C comprises
suppressing the expression of HLA-C on cell surfaces.

22. The method, according to claim 20, which comprises the use of a compound that is directed against HLA-C, wherein said compound is
an anti-HLA-C antibody or fragment thereof;
a small molecule inhibitor; or
a small interfering RNA.

23. The peptide-HLA-C complex, according to claim 6, wherein the HLA-C is HLA-C*06:02.

24. The method, according to claim 20, wherein the HLA-C is HLA-C* 06.02.

25. The method, according to claim 21, wherein suppressing the expression of HLA-C comprises suppressing the expression of HLA-C on cell surfaces by small interfering RNAs or other molecule that reduces HLA-C transcription, HLA-C translation or HLA-C transport to the cell surface.


SEQUENCE LISTING (78 sequences)

SEQ ID NO: 1 - protein, 9 aa, Artificial Sequence
  Antigenic peptide
FRSYRTRRL

SEQ ID NO: 2 - protein, 9 aa, Artificial Sequence
  Antigenic peptide
FRHRRTLRL

SEQ ID NO: 3 - protein, 9 aa, Artificial Sequence
  Antigenic peptide
FRCRQYRRL

SEQ ID NO: 4 - protein, 9 aa, Artificial Sequence
  Antigenic peptide
FRWYRVRRL

SEQ ID NO: 5 - protein, 9 aa, Artificial Sequence
  Antigenic peptide
RRNWNYRRL

SEQ ID NO: 6 - protein, 9 aa, Artificial Sequence
  Antigenic peptide
GRASRTRRL

SEQ ID NO: 7 - protein, 9 aa, Artificial Sequence
  Antigenic peptide
GRNARVRRL

SEQ ID NO: 8 - protein, 9 aa, Artificial Sequence
  Antigenic peptide
MRSHRYLRL

SEQ ID NO: 9 - protein, 9 aa, Artificial Sequence
  Antigenic peptide
RRYFRCLRM

SEQ ID NO: 10 - protein, 9 aa, Homo sapiens
ARHRRSLRL

SEQ ID NO: 11 - protein, 9 aa, Homo sapiens
RRQRRSRRL

SEQ ID NO: 12 - protein, 8 aa, Homo sapiens
RQRRSRRL

SEQ ID NO: 13 - protein, 9 aa, Homo sapiens
ARSVRTRRL

SEQ ID NO: 14 - protein, 9 aa, Homo sapiens
SRASRALRL

SEQ ID NO: 15 - protein, 9 aa, Homo sapiens
LRAGRSRRL

SEQ ID NO: 16 - protein, 8 aa, Homo sapiens
RAGRSRRL

SEQ ID NO: 17 - protein, 9 aa, Homo sapiens
VRSRRCLRL

SEQ ID NO: 18 - protein, 8 aa, Homo sapiens
RSRRCLRL

SEQ ID NO: 19 - protein, 9 aa, Homo sapiens
RRFCRTSRM

SEQ ID NO: 20 - protein, 9 aa, Homo sapiens
HRHSRILRL

SEQ ID NO: 21 - protein, 9 aa, Homo sapiens
SRAVRALRL

SEQ ID NO: 22 - protein, 2969 aa, Homo sapiens
MDPRNTAMLGLGSDSEGFSRKSPSAISTGTLVSKREVELEKNTKEEEDLRKRNRERNIEAGKDDGLTDAQQQFSVKETNFSEGNLKLKIGLQAKRTKKPPKNLENYVCRPAIKTTIKHPRKALKSGKMTDEKNEHCPSKRDPSKLYKKADDVAAIECQSEEVIRLHSQGENNPLSKKLSPVHSEMADYINATPSTLLGSRDPDLKDRALLNGGTSVTEKLAQLIATCPPSKSSKTKPKKLGTGTTAGLVSKDLIRKAGVGSVAGIIHKDLIKKPTISTAVGLVTKDPGKKPVFNAAVGLVNKDSVKKLGTGTTAVFINKNLGKKPGTITTVGLLSKDSGKKLGIGIVPGLVHKESGKKLGLGTVVGLVNKDLGKKLGSTVGLVAKDCAKKIVASSAMGLVNKDIGKKLMSCPLAGLISKDAINLKAEALLPTQEPLKASCSTNINNQESQELSESLKDSATSKTFEKNVVRQNKESILEKFSVRKEIINLEKEMFNEGTCIQQDSFSSSEKGSYETSKHEKQPPVYCTSPDFKMGGASDVSTAKSPFSAVGESNLPSPSPTVSVNPLTRSPPETSSQLAPNPLLLSSTTELIEEISESVGKNQFTSESTHLNVGHRSVGHSISIECKGIDKEVNDSKTTHIDIPRISSSLGKKPSLTSESSIHTITPSVVNFTSLFSNKPFLKLGAVSASDKHCQVAESLSTSLQSKPLKKRKGRKPRWTKVVARSTCRSPKGLELERSELFKNVSCSSLSNSNSEPAKFMKNIGPPSFVDHDFLKRRLPKLSKSTAPSLALLADSEKPSHKSFATHKLSSSMCVSSDLLSDIYKPKRGRPKSKEMPQLEGPPKRTLKIPASKVFSLQSKEEQEPPILQPEIEIPSFKQGLSVSPFPKKRGRPKRQMRSPVKMKPPVLSVAPFVATESPSKLESESDNHRSSSDFFESEDQLQDPDDLDDSHRPSVCSMSDLEMEPDKKITKRNNGQLMKTIIRKINKMKTLKRKKLLNQILSSSVESSNKGKVQSKLHNTVSSLAATFGSKLGQQINVSKKGTIYIGKRRGRKPKTVLNGILSGSPTSLAVLEQTAQQAAGSALGQILPPLLPSSASSSEILPSPICSQSSGTSGGQSPVSSDAGFVEPSSVPYLHLHSRQGSMIQTLAMKKASKGRRRLSPPTLLPNSPSHLSELTSLKEATPSPISESHSDETIPSDSGIGTDNNSTSDRAEKFCGQKKRRHSFEHVSLIPPETSTVLSSLKEKHKHKCKRRNHDYLSYDKMKRQKRKRKKKYPQLRNRQDPDFIAELEELISRLSEIRITHRSHHFIPRDLLPTIFRINFNSFYTHPSFPLDPLHYIRKPDLKKKRGRPPKMREAMAEMPFMHSLSFPLSSTGFYPSYGMPYSPSPLTAAPIGLGYYGRYPPTLYPPPPSPSFTTPLPPPSYMHAGHLLLNPAKYHKKKHKLLRQEAFLTTSRTPLLSMSTYPSVPPEMAYGWMVEHKHRHRHKHREHRSSEQPQVSMDTGSSRSVLESLKRYRFGKDAVGERYKHKEKHRCHMSCPHLSPSKSLINREEQWVHREPSESSPLALGLQTPLQIDCSESSPSLSLGGFTPNSEPASSDEHTNLFTSAIGSCRVSNPNSSGRKKLTDSPGLFSAQDTSLNRLHRKESLPSNERAVQTLAGSQPTSDKPSQRPSESTNCSPTRKRSSSESTSSTVNGVPSRSPRLVASGDDSVDSLLQRMVQNEDQEPMEKSIDAVIATASAPPSSSPGRSHSKDRTLGKPDSLLVPAVTSDSCNNSISLLSEKLTSSCSPHHIKRSVVEAMQRQARKMCNYDKILATKKNLDHVNKILKAKKLQRQARTGNNFVKRRPGRPRKCPLQAVVSMQAFQAAQFVNPELNRDEEGAALHLSPDTVTDVIEAVVQSVNLNPEHKKGLKRKGWLLEEQTRKKQKPLPEEEEQENNKSFNEAPVEIPSPSETPAKPSEPESTLQPVLSLIPREKKPPRPPKKKYQKAGLYSDVYKTTDPKSRLIQLKKEKLEYTPGEHEYGLFPAPIHVVFFVSGKYLRQKRIDFQLPYDILWQWKHNQLYKKPDVPLYKKIRSNVYVDVKPLSGYEATTCNCKKPDDDTRKGCVDDCLNRMIFAECSPNTCPCGEQCCNQRIQRHEWVQCLERFRAEEKGWGIRTKEPLKAGQFIIEYLGEVVSEQEFRNRMIEQYHNHSDHYCLNLDSGMVIDSYRMGNEARFINHSCDPNCEMQKWSVNGVYRIGLYALKDMPAGTELTYDYNFHSFNVEKQQLCKCGFEKCRGIIGGKSQRVNGLTSSKNSQPMATHKKSGRSKEKRKSKHKLKKRRGHLSEEPSENINTPTRLTPQLQMKPMSNRERNFVLKHHVFLVRNWEKIRQKQEEVKHTSDNIHSASLYTRWNGICRDDGNIKSDVFMTQFSALQTARSVRTRRLAAAEENIEVARAARLAQIFKEICDGIISYKDSSRQALAAPLLNLPPKKKNADYYEKISDPLDLITIEKQILTGYYKTVEAFDADMLKVFRNAEKYYGRKSPVGRDVCRLRKAYYNARHEASAQIDEIVGETASEADSSETSVSEKENGHEKDDDVIRCICGLYKDEGLMIQCDKCMVWQHCDCMGVNSDVEHYLCEQCDPRPVDREVPMIPRPHYAQPGCVYFICLLRDDLLLRQGDCVYLMRDSRRTPDGHPVRQSYRLLSHINRDKLDIFRIEKLWKNEKEERFAFGHHYFRPHETHHSPSRRFYHNELFRVPLYEIIPLEAVVGTCCVLDLYTYCKGRPKGVKEQDVYICDYRLDKSAHLFYKIHRNRYPVCTKPYAFDHFPKKLTPKKDFSPHYVPDNYKRNGGRSSWKSERSKPPLKDLGQEDDALPLIEEVLASQEQAANEIPSLEEPEREGATANVSEGEKKTEESSQEPQSTCTPEERRHNQRERLNQILLNLLEKIPGKNAIDVTYLLEEGSGRKLRRRTLFIPENSFRK

SEQ ID NO: 23 - protein, 481 aa, Homo sapiens
MGKLRPGRVEWLASGHTERPHLFQNLLLFLWALLNCGLGVSAQGPGEWTPWVSWTRCSSSCGRGVSVRSRRCLRLPGEEPCWGDSHEYRLCQLPDCPPGAVPFRDLQCALYNGRPVLGTQKTYQWVPFHGAPNQCDLNCLAEGHAFYHSFGRVLDGTACSPGAQGVCVAGRCLSAGCDGLLGSGALEDRCGRCGGANDSCLFVQRVFRDAGAFAGYWNVTLIPEGARHIRVEHRSRNHLALMGGDGRYVLNGHWVVSPPGTYEAAGTHVVYTRDTGPQETLQAAGPTSHDLLLQVLLQEPNPGIEFEFWLPRERYSPFQARVQALGWPLRQPQPRGVEPQPPAAPAVTPAQTPTLAPDPCPPCPDTRGRAHRLLHYCGSDFVFQARVLGHHHQAQETRYEVRIQLVYKNRSPLRAREYVWAPGHCPCPMLAPHRDYLMAVQRLVSPDGTQDQLLLPHAGYARPWSPAEDSRIRLTARRCPG

SEQ ID NO: 24 - protein, 416 aa, Homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSSLYIILSTGGIFLLVTLVTVCACWKPSKRKQKKLEKQNSLEYMDQNDDRLKPEADTLPRSGEQERKNPMALYILKDKDSPETEENPAPEPRSATEPGPPGYSVSPAVPGRSPGLPIRSARRYPRSPARSPATGRTHSSPPRAPSSPGRSRSASRTLRTAGVHIIREQDEAGPVEISA

SEQ ID NO: 25 - protein, 369 aa, Homo sapiens
MWCLERLRLGPECLRRSGDWLLPGRARGAKSRTTAACANVLTPDRIPEFCIPPRLMPRLALAALRNSWVEEAGMDEGAGRTDWDPRSQAALSLPHLPRVRTAYGFCALLESPHTRRKESLLLGGPPAPRPRAHTYGGGGGPDALLGTLRVPRAPGPATPAAPGCPRPPQDALARRPRGCRLLRVPDGLLSRALRAGRSRRLTRVRSVSSGNEDKERRAGSQSPARAPSTSPPSSRVPFPERLEAEGTVALGRAGDALRLAAEYCPGTGRLRLRLLRAESPAGGAPGPRAVSCRLSLVLRPPGTALRQCSTVVGRSRKASFDQDFCFDGLSEDEVRRLAVRVKARDEGRGRERGRLLGQGELSLGALLLL

SEQ ID NO: 26 - protein, 364 aa, Homo sapiens
MRLLEKLCSSAAGSSAPKPAFAKVLTPNRIPEFCIPPRLPAPCTLESPIRAAAVPRRCAAESDLWPRAADEDAGRTDWDPRSQAALSLPHLPRVRTTYGFCALLESPHTRRKESLLLGGPPAPRPRAHSCGGGGGPDAPLGTLCGPRGPGPATPAAPGGPRLPQDALAAGPRRCRLLRVPDGLLSRALRAGRSRRLARVRSVSSGNEDEERRAGSESPARAPSSSPLSSRAPLPERLEAKGTVALGRAGDALRLAAEYCPGTRRLRLRLLRAESLFGGAPGPRAVRCRLSLVLRPPGTARWQCSAVVGRSRKASFDQDFCFDGLSEDEVRRLAVRVKARDEGRGRDRGRLLGQGELSLGALLLL

SEQ ID NO: 27 - protein, 208 aa, Homo sapiens
MLGLLVALLALGLAVFALLDVWYLVRLPCAVLRARLLQPRVRDLLAEQRFPGRVLPSDLDLLLHMNNARYLREADFARVAHLTRCGVLGALRELRAHTVLAASCARHRRSLRLLEPFEVRTRLLGWDDRAFYLEARFVSLRDGFVCALLRFRQHLLGTSPERVVQHLCQRRVEPPELPADLQHWISYNEASSQLLRMESGLSDVTKDQ

SEQ ID NO: 28 - protein, 507 aa, Homo sapiens
MDPSEKKISVWICQEEKLVSGLSRRTTCSDVVRVLLEDGCRRRRRQRRSRRLGSAGDPHGPGELPEPPNEDDEDDDEALPQGMLCGPPQCYCIVEKWRGFERILPNKTRILRLWAAWGEEQENVRFVLVRSEASLPNAGPRSAEARVVLSRERPCPARGAPARPSLAMTQEKQRRVVRKAFRKLAKLNRRRQQQTPSSCSSTSSSTASSCSSSPRTHESASVERMETLVHLVLSQDHTIRQQVQRLHELDREIDHYEAKVHLDRMRRHGVNYVQDTYLVGAGIELDGSRPGEEPEEVAAEAEEAAAAPPLAGEAQAAALEELARRCDDLLRLQEQRVQQEELLERLSAEIQEELNQRWMRRRQEELAAREEPLEPDGGPDGELLLEQERVRTQLSTSLYIGLRLNTDLEAVKSDLDYSQQQWDSKKRELQGLLQTLHTLELTVAPDGAPGSGSPSREPGPQACADMWVDQARGLAKSGPGNDEDSDTGLSSMHSQDSDSLPMCESLV

SEQ ID NO: 29 - protein, 51 aa, Homo sapiens [sequence omitted]

SEQ ID NO: 30 - protein, 101 aa, Homo sapiens
GCETPRLPEIALRALPRPGVSSLGTILCVPSVPVGPIVLTSATPESSRFTRPAPSTVPEAHRHSRILRLAPVPCRCYRPPAPAHTSRSTSLFYESPSTFNV

SEQ ID NO: 31 - protein, 356 aa, Homo sapiens
MGLMVRGGVTCRSQERLLGVGTGTASAQPPVTASPYCSFTGRSRADAPARAAGNGQPRLRAQVDLSSASSVLVTVLDAKALPLTLLSWGSLCATAQNHNNQEGAPAQPKGSMANQRALRFPPPSGSPPGLHTRMRLTSPPQPHFEPPPPTTVGAPRSLQGDWFGSDSRAVRALRLIGWASRSLHPLPGSRDRAHPAAEEEDDPDRPIEFSSSKANPHRWSVGHTMGKGHQRPWWKVLPLSCFLVALIIWCYLREESEADQWLRQEGSRQPPFGFDVTFARDCPGYACVLSTEGLGWWMGHLAMLIRVKAEQNLSRSETAPRLALDVQGFHRQDFSDPWGRFQLHCMLLDLPSLCIT

SEQ ID NO: 32 - protein, 9 aa, Artificial Sequence
  Antigenic peptide
VRCWRGRRL

SEQ ID NO: 33 - protein, 9 aa, Artificial Sequence
  mutated peptide ligands of the protein ADAMTL5
VRSRRCLRM

SEQ ID NO: 34 - protein, 9 aa, Artificial Sequence
  mutated peptide ligands of the protein ADAMTL5
VRSRRCLRT

SEQ ID NO: 35 - protein, 9 aa, Artificial Sequence
  mutated peptide ligands of the protein ADAMTL5
VRSRRCLRV

SEQ ID NO: 36 - protein, 9 aa, Artificial Sequence
  mutated peptide ligands of the protein ADAMTL5
VRSRRCLRY

SEQ ID NO: 37 - protein, 9 aa, Artificial Sequence
  mutated peptide ligands of the protein ADAMTL5
ARSRRCLRM

SEQ ID NO: 38 - protein, 9 aa, Artificial Sequence
  mutated peptide ligands of the protein ADAMTL5
VRARRCLRT

SEQ ID NO: 39 - protein, 9 aa, Artificial Sequence
  mutated peptide ligands of the protein ADAMTL5
VRSARCLRV

SEQ ID NO: 40 - protein, 9 aa, Artificial Sequence
  mutated peptide ligands of the protein ADAMTL5
VRSRRALRY

SEQ ID NO: 41 - protein, 9 aa, Serpula lacrymans var. lacrymans S7.9
HRTRRTRRL

SEQ ID NO: 42 - protein, 9 aa, Mycobacterium tuberculosis
RRTRRTRRL

SEQ ID NO: 43 - protein, 9 aa, Chlamydia trachomatis
FRSYRVRRL

SEQ ID NO: 44 - protein, 9 aa, Aspergillus niger
GRASRSRRL

SEQ ID NO: 45 - protein, 9 aa, Klebsiella pneumoniae
KRSRRVLRL

SEQ ID NO: 46 - protein, 9 aa, Nocardia brasiliensis
RRTRRSRRL

SEQ ID NO: 47 - protein, 9 aa, Pseudomonas syringae
MRWFRVRRL

SEQ ID NO: 48 - protein, 9 aa, Pectobacterium atrosepticum
WRHSRALRL

SEQ ID NO: 49 - protein, 9 aa, Burkholderia lata
GRTSRTRRL

SEQ ID NO: 50 - protein, 8 aa, Desulfovibrio aminophilus
RASRTRRL

SEQ ID NO: 51 - protein, 8 aa, Flavonifractor plautii
RAARTRRL

SEQ ID NO: 52 - protein, 9 aa, Coffea canephora
YRSYRTRRM

SEQ ID NO: 53 - protein, 9 aa, Malus domestica Golden Delicious
ARSFRSLRL

SEQ ID NO: 54 - protein, 9 aa, Malus domestica Golden Delicious
ARSSRSLRL

SEQ ID NO: 55 - protein, 9 aa, Spinacia oleracea
HRNHRYLRL

SEQ ID NO: 56 - protein, 9 aa, Triticum aestivum
RRPERRRRL

SEQ ID NO: 57 - protein, 9 aa, Triticum aestivum
RRPGRRRRL

SEQ ID NO: 58 - protein, 29 aa, homo sapiens
MKRERGALSRASRALRLAPFVYLLLIQTD

SEQ ID NO: 59 - protein, 121 aa, homo sapiens
AGPRRCRILRVPDGLLSRALRAGRSRRLARVRSVSSGNEDEERRAGSESPARAPSSSPLSSRAPLPERLEAKGTVALGRAGDALRLAAEYCPGTRRLRLRLLRAESLVGGAPGPRAVRCRL

SEQ ID NO: 60 - protein, 81 aa, Serpula lacrymans var. lacrymans S7.9
MPPYCGPSPGLLLQTQVQVNNNSDDDILEEDGSTLAPSAVDSSMGWLEIGGYHTYVHRTRRTRRLTHSKISHHFNGLISAA

SEQ ID NO: 61 - protein, 1435 aa, Mycobacterium tuberculosis
  misc_feature (1200)..(1200): Xaa can be any naturally occurring amino acid
MTGSISGEADLRHWLIDYLVTNIGCTPDEVDPDLSLADLGVSSRDAVVLSGELSELLGRTVSPIDFWEHPTINALAAYLAAPEPSPDSDAAVKRGARNSLDEPIAVVGMGCRFPGGISCPEALWDFLCERRSSISQVPPQRWQPFEGGPPEVAAALARTTRWGSFLPDIDAFDAEFFEISPSEADKMDPQQRLLLEVAWEALEHAGIPPGTLRRSATGVFAGACLSEYGAMASADLSQVDGWSNSGGAMSIIANRLSYFLDLRGPSVAVDTACSSSLVAIHLACQSLRTQDCHLAIAAGVNLLLSPAVFRGFDQVGALSPTGQCRAFDATADGFVRGEGAGVVVLKRLTDAQRDGDRVLAVICGSAVNQDGRSNGLMAPNPAAQMAVLRAAYTNAGMQPSEVDYVEAHGTGTLLGDPIEARALGTVLGRGRPEDSPLLIGSVKTNLGHTEAAAGIAGFIKTVLAVQHGQIPPNQHFETANPHIPFTDLRMKVVDTQTEWPATGHPRRAGVSSFGFGGTNAHVVIEQGQEVRPAPGQGLSPAVSTLVVAGKTMQRVSATAGMLADWMEGPGADVALADVAHTLNHHRSRQPKFGTVVARDRTQAIAGLRALAAGQHAPGVVNPAEGSPGPGTVFVYSGRGSQWAGMGRQLLADEPAFAAAVAELEPVFVEQAGFSLHDVLANGEELVGIEQIQLGLIGMQLALTELWCSYGVRPDLVIGHSMGEVAAAVVAGALTPAEGLRVTATRSRLMAPLSGQGGMALLELDAPTTEALIADFPQVTLGIYNSPRQTVIAGPTEQIDELITRVRARDRFASRVNIEVAPHNPAMDALQPAMRSELADLTPRTPTIGIISTTYADLHTQPVFDAEHWATNMRNPVHFQQAIASAGSGADGAYHTFIEISAHPLLTQAIIDTLHSAQPGARYTSLGTLQRDTDDVVTFRTNLNKAHTIHPPHTPHPPEPHPPIPTTPWQHTRHWITTKYPAGSVGSAPRAGTLLGQHTTVATVSASPPSHLWQARLAPDAKPYQGGHRFHQVEVVPASVVLHTILSAATELGYSALSEVRFEQPIFADRPRLIQVVADNRAISLASSPAAGTPSDRWTRHVTAQLSSSPSDSASSLNEHHRANGQPPERAHRDLIPDLAELLAMRGIDGLPFSWTVASWTQHSSNLTVAIDLPEALPEGSTGPLLDAAVHLAALSDVADXRCRSPRRRRRRRRNRRTCRRYRRPRRDHHLGYRCRRCRRRRQNRPTRRYRRRRRRRRTRRTRRLRRPHRCRIAGRHYRRDHRGQRQWRLRLHRRPRPSRRPCRRRRPRRRRRRQHCRSHRARRPRRRQTARRCRRRRRSHRCRRSHRRRPSRRPGRTARRPRQPRRSHRCRRSRRRRRHRRRRTAHRPRPHRRCHRRPGRRRRGRRRLRRRPRPRRTAHRRRRPRRHPQRHQERW

SEQ ID NO: 62 - protein, 80 aa, Chlamydia trachomatis
MKIQIEQLFRSYRVRRLTEQDVASILELERGNSLYYQCSPPAPSQASVVEDMQALPVGKIMADKYFLEFFQGRALVTIVT

SEQ ID NO: 63 - protein, 387 aa, Aspergillus niger
ATPRKASNRPKKRTLVRWDGMSLSRLQQSHLVEKLVRPRLTSIENLNELLLLTVQSVCNTKSIKIPWAEVANTMGHNVTEGAIVQHLAKLRSRRVSADKAVPPPLRRGATGMSKSSEETPTKSVADEAPNTRASSSKNNFSGSRARPRYQDTSSDEEWVQGRASRSRRLQKRRKSAVKAEESSSEVEKSESDDEDKCSSVASSDELLVPGAKFLEYPNDVGQPQRKVVTLKYRRPSATEADNAPSKSLDTANDTTGVNTSVVVSSKQPSGVYSFDNDPHMMDYFPHDVPSSYVPGNTSNELPELPSGENISSGDLWDTDLCPPFSSGMVYSGYNPDFNAIAANDPLAFSQDMTSSLSYEPGSYPNMPISYTRSDGPLNDFDMLEDMK

SEQ ID NO: 64 - protein, 213 aa, Klebsiella pneumoniae
MSDNDAIFHELDQVFSAILAGASPAGRQRTARSVGTMLRRSQSQRIGKQTAPDGTLYPKRSRRVLRLQAGIGFIWQGEKRRLRNWRAVRGSRGRMLTGFDEDRGAVRSFYTEDIERYLNISFNERRQNITKPDPMFRRLRTARFLKVSATPGGAEVGFSGVAARIARVHQFGLRDKVNDSGAMATYPRRELLGLSKADRMAIARQVIDSLGMR

SEQ ID NO: 65 - protein, 168 aa, Nocardia brasiliensis
ATGCTTGATATTGCCTACCGAAAGTCCGCGATCGACCAGGACACGCAGCGGCTCGATATCAGTCGATATCTGGCCTGGGGCAACGTCGCAGATCCGGTCCGCCGCACGCGGCGGTCCAGGCGCCTCGCCGACGATCTCGACCAGTCCGAGCACAAGACAAGTGCCTGA

SEQ ID NO: 66 - protein, 1219 aa, Pseudomonas syringae
MLASSGRILAQVKSRRGHRGPFPIGEIVGFLTTLWNRTTEQPEDSYLLILEREPAAWPNSKDIAWLHELTVLMAALPPGVSSELLDRTRIYVLPNPRWAAHQVIEARRECSALEADAYYGVVLLRAGNLADANGMRKSGGFLGLSPSDVELEINTLAPMFTSARIQAALSRGLCEAVDFSTPLEEADFFSGVDVQPGHVVAGLLVERTVLRESVVTALTEHRNILLKGPSGAGKSGLQWDVAYSLRHSMRWFRVRRLAVEDIVDLVALANNCCASVDAPVGFLMDNLGAGLMDGWDALAREVAIQPSLALLASIREEDIYPLVERHKAVEVRVRTDEDFAEQFWRELRERDKTEWTNWLEPWTASRGLLLEYAHILTKGSRLDETLRQQVAVRATDSTRHIELEILGLCAAVTSIGARVDVARLPEVLGRSAIEIGTSLPRIVNEHLMRDLGDGSMGGAHELRSQHLLGEIGHISLRAESETFELALGTVLEADIGIFLARTLERHPVLEDALLDAIAEKLVQRPSARLMTVVLNGLGEREIAQVIDAWLSSPEVAEVPLSQISLVALFGTADVELPDLPEFKLAASAIRLMAQMKAQSTHSSLRGRLLRRLSISDLQRMLEQVNDIATLNTLLVAHVGHALTPTLRDALMEMSPGLLAGELSQVSALLGTAHVLDPEVAAQWVSKTGELALLRRIGSEVPWATEASVQSLPEGLAAICDIRTAPARYQPDPHGSVVTLCELLLALCPAADLAIARAVTVSGELLCYGGMPVADKKIPRSNLPPNSLPAWNRRWGNAIAARLAPESYGAYLGQAADDIKQLNSSLFDFFDGLFRNSPSSAALEILGNVHERSRSMPSPNLPWFAKCASGESVGVKTSNLQQILFDCSTDMVRRFVRLPADSQSYIGWIASVLKRIQDSIELEPWQITAYGTQAKAALESLATLIDDLRALAGDGLQRGMNPAVMYRRLRAPKGQALTEARASARKRAKSGKALKERDLQRLFRALTSSARVYVKSSKDELLMWPLLEVLVTFELEQIQRWPSFAHEGWAIWRSLIENALHLTVMPVVNGMCVPMCAIGGHSEPFPSEPKAWEWIHAVGLRAVPEIASRMWGIFSDAVMDVAALEAQGFGLDNSPEIELLARIDARARLRISKASIDERLPTPLRIMTDSLVELVDQQPWLLYNAQLAALRGEIDEIGEILMDALYTCYSVDLRATPTLT

SEQ ID NO: 67 - protein, 285 aa, Pectobacterium atrosepticum
MLKYRLITAFILIPIVIAALFLLPPLGFTLVTLAVCMLAAWEWGQLAGFASYGQRLWLAILCGFLLALMLLSLPAYHYSVHIPQISIALWSSLVWWGIALFLVLFYPASASFWRHSRALRLVFGIMTIVPFFWGMVALRHYNYAINPFAGAWWLLYVMLLVWGADSGAYMFGKLFGKRKLAPKVSPGKTWEGFLGGLATSALISLLFSLYAPLTVAASTLLICSIAAALASVLGDLTESMFKREAGIKDSSHLIPGHGGVLDRIDSLTAAVPVFSCLMLLLFKVA

SEQ ID NO: 68 - protein, 59 aa, Burkholderia lata
MRSGRTSRTRRLGDSIRRPDYAAYGELDTSALLLYCSAQFAPDRPAPEPPAPWPRGGAT

SEQ ID NO: 69 - protein, 106 aa, Desulfovibrio aminophilus
MDGGPSLLFTLTALLSGFCAGGWLAWKINQAHIDRAPGDGYREKLRASRTRRLIGAAGALAVLGVSLLFPVQSLVITSACIGYALTTLLHPRPNGEHPGPGRDGGA

SEQ ID NO: 70 - protein, 84 aa, Flavonifractor plautii
MAERKSEQERIKQIDQQLAELKAKRRALAVMAAKKERAARTRRLIQIGALSEKYLDCYNISVENYEILISRIVNLPAVKELLNT

SEQ ID NO: 71 - protein, 526 aa, Coffea canephora
MEVEAHALSSFDLNSSPDNFPFTTTAAREPPELRNSQPFRSSKLLGYSESDHAPVREDLGSPVAAGGKCQDVGGNSASPVANAFAGEEPLPEKMGRCPSRAAMKLQKVYRSYRTRRMLADCAVVAEELWWQALDFARLNHSTISFFNFSKPESAASRWNRVSLNASKVGKGLSKDAKAQKLAFQHWIEAIDPRHRYGHSLHLYYEEWCKGDAGQPFFFWLDLGDGKEVDLTACPRSKLRKQHIKYLGPKEREHYEYLVTEGKIWHKQSGKPLDTTEASPGAKWIFVMSTSKRLYAGEKKKGTFHHSSFLAGGVTLAAGRLVVIDGTLQSISPYSGHYRPTDDSFDTFLDFLKAHGVNLDEVEMNKANEDYENFEEAKTTKDESTSEVSTVSDSCETNAPDDGAVETIEAPKLEINVNYQRTLSGGLQSPKAEVPKTSILQRINSKKAARSYQLGKQLSLKWSTGAGPRIGCIADYPIELRIQALELTNLSPRLPSTSATPIWGTLPSPIRLPSQELTNGDLKLSGI

SEQ ID NO: 72 - protein, 142 aa, Malus domestica
  misc_feature (39)..(39): Xaa can be any naturally occurring amino acid
  misc_feature (60)..(60): Xaa can be any naturally occurring amino acid
  misc_feature (86)..(86): Xaa can be any naturally occurring amino acid
MYRLARHCCWRLQAFWDLISLPYEHQISLAPQIKFEINXSHSLKFGFAFRSNSSPSPFHXYPFCDQQRVYSVSFDAFRLSVSARSXRSLRLSRCIYFSPGFTPNIEKLNFGGIENFEAINAQKFRNPNLEGLDRRNFINLIN

SEQ ID NO: 73 - protein, 142 aa, Malus domestica; identical to SEQ ID NO: 72
  misc_feature (39)..(39): Xaa can be any naturally occurring amino acid
  misc_feature (60)..(60): Xaa can be any naturally occurring amino acid
  misc_feature (86)..(86): Xaa can be any naturally occurring amino acid

SEQ ID NO: 74 - protein, 483 aa, Spinacia oleracea
MSIALHRNHRYLRLPYQLITSIRHYSRISPPRPTGPDQVIRVVNNIAQLGSPKEGPKPRQLLSLPPFPSDPLPDKSSTKCLNGNQRTRVTAISWVKYYFDEVHDSVIQSHFNKGLVQIEYPSMKGSSSTQGKKSIRKIKHNEPMKAGAKIHVPVSIAESRISRRFDPIPCGTLYPNADEIAYLQRLVMYKDSAIIVLNKPPKLPVKGNLPIHNSMDALAAAALSYDYEEGPQLVHRLDRESSGILLMGRTKESTSYLQQLFSNMNVAKTSCLAWNDACGATFMKYWALVIGSPREKEGVICAPLTKVLLDDGKTERVMLAHPSGLEASQEAMTEYRVLGPMINGCSWIELRPHTSRKHQLRVHCAEALGTPIVGDYKYGWFVHQKWKQTPQVNIEPISGEPYKLRRPDGLDVQKGSVLSKVPLLHLHCRELVLPNVAKFLGVLGQKSSDYRPKLSEKPNVLRVVASMPSHMKISWNLMSSYLV

SEQ ID NO: 75 - protein, 100 aa, Triticum aestivum
  misc_feature (2)..(2): Xaa can be any naturally occurring amino acid
  misc_feature (14)..(14): Xaa can be any naturally occurring amino acid
GXIGTATDKPRRPXRRRRLTPVRPGRREWASHGRSSPTSTPSSGQASRCCTLCMCRCAPWRARPRWTTSSGWPTGSSTHSSPSSRCSPSPSSTGYGCGTQ

SEQ ID NO: 76 - protein, 101 aa, Triticum aestivum
  misc_feature (3)..(3): Xaa can be any naturally occurring amino acid
  misc_feature (15)..(15): Xaa can be any naturally occurring amino acid
RGXIGTATDKPRRPXRRRRLTPVRPGRREWASHGRSSPTSTPSSGQASRCCTLCMCRCAPWRARPRWTTSSGWPTGSSTHSSPSSRCSPSPSSTGYGCGTQ

SEQ ID NO: 77 - DNA, 37 bp, Artificial Sequence
  Primer
caccatggtgcggagccggcggtgcctgcggctgtga

SEQ ID NO: 78 - DNA, 30 bp, Artificial Sequence
  Primer
tcacagccgcaggcaccgccggctccgcac